SEQUENCE LISTING (10 sequences)

SEQ ID NO: 1 - protein, 255 aa, Lactobacillus delbrueckii
MAFGRKKHLNNDTMKNGVKLITLANPQSVISEQFRNIRTYINFMNVDREVKTIVFTSAMASAGKSTVSANVAITMAQAGKKTILVDADLRRPTMHSTFNVSNSNGLTTLLTSRSMEMDANSVIRESGVENLSILTAGPIPPNPSELLSSKHMLDLIEDLKQEYDMVVLDLAPILDAGETQQLTSSLDGTILVVRQAHSQKSAVKRAVELLKLTKSPILGYVMNDVDADGDDGYGYGYGYGYGEEDEKKGLFGRKK

SEQ ID NO: 2 - DNA, 768 bp, Lactobacillus delbrueckii
atggcattcggtcgaaagaagcatttaaacaacgatacgatgaagaacggggtcaagctgattactttggccaacccgcaaagcgttatttcagaacagttcagaaacatccgaacctatatcaattttatgaacgttgatagggaagtaaagacgattgtctttacttcagccatggccagcgccggcaagtcgactgtcagtgctaatgtagccattaccatggcgcaagccggcaagaagacgatcttggtcgatgcggacttgcgccggccaactatgcactctacttttaacgtatctaatagcaatggcttgacaactttgttgactagccggtctatggaaatggacgcaaatagcgtgatccgggaaagtggtgtagagaacctgtcaatcttgacggcaggtccgattccgccaaacccatcagaacttctgtcttccaagcatatgttggatttgattgaagatttaaagcaagaatatgatatggtcgtgcttgacttagcaccgatcttggacgcgggcgaaacccagcaactgaccagttctttggacgggacgatcttggttgtgcgccaggcacattcacagaagtcagcagttaagcgggcagttgagctgcttaagctgactaagtcaccaatcttgggttatgttatgaacgatgttgatgccgatggggatgacggctatggatatggttatggctacggttatggcgaagaagatgagaagaaggggctctttgggagaaagaagtag

SEQ ID NO: 3 - DNA, 18 bp, Lactobacillus delbrueckii
gggctcgctattctctga

SEQ ID NO: 4 - protein, 5 aa, Lactobacillus delbrueckii
GLAIL

SEQ ID NO: 5 - DNA, 21 bp, Lactobacillus delbrueckii
ggctcgctattctctgattga

SEQ ID NO: 6 - protein, 6 aa, Lactobacillus delbrueckii
GSLFSD

SEQ ID NO: 7 - DNA, 32 bp, Artificial Sequence
  Lb_epsC_rec1_F
caggagctcaggtgaaatcgcacatatcacga

SEQ ID NO: 8 - DNA, 31 bp, Artificial Sequence
  Lb_epsC_rec1_R
gacgagctctcaactaccggcatgagctact

SEQ ID NO: 9 - DNA, 28 bp, Artificial Sequence
  Lb_epsF_rec1_F
ccggtcgacacaatcggtgttatcttcc

SEQ ID NO: 10 - DNA, 29 bp, Artificial Sequence
  Lb_epsF_rec1_R
cgggtcgacgatcataggctttagacact

(19) United States
(12) Patent Application Publication
(10) Pub. No.: US 2023/0404093 A1
(43) Pub. Date: Dec. 21, 2023

(54) PROTEIN HAVING FERMENTED MILK VISCOSITY IMPROVING EFFECT, FERMENTED MILK USING THE SAME AND METHOD FOR PRODUCING THE SAME

(71) Applicant: MEIJI CO., LTD., Tokyo (JP)

(72) Inventors: Shin FUJIWARA, Tokyo (JP); Yoshiko HONME, Tokyo (JP); Yasuko SASAKI, Kanagawa (JP); Hiromu KUDO, Kanagawa (JP)

(73) Assignee: MEIJI CO., LTD., Tokyo (JP)

(21) Appl. No.: 18/031,301

(22) PCT Filed: Oct. 8, 2021
(86) PCT No.: PCT/JP2021/037285

§ 371 Date: Apr. 11, 2023

(30) Foreign Application Priority Data
Oct. 12, 2020 (JP) 2020-172077

(51) Int. Cl. A23C 9/123; C07K 14/335; A23C 9/137; C12Q 1/04; C12P 19/04; C12N 15/74

(52) U.S. Cl. CPC A23C 9/1238; C07K 14/335; A23C 9/137; C12R 2001/225; C12P 19/04; C12N 15/746; A23C 2220/202; C12Q 1/04

(57) ABSTRACT

The present invention provides at least one protein selected from the group consisting of the following proteins (a) to (d):
(a) a protein which is composed of an amino acid sequence set forth in SEQ ID NO: 1;
(b) a protein which is, in the amino acid sequence set forth in SEQ ID NO: 1, composed of an amino acid sequence in which one or more amino acids other than tyrosine at position 40 are substituted, deleted, inserted, and/or added and has a fermented milk viscosity improving effect;
(c) a protein which is composed of an amino acid sequence having 80% or more identity with the amino acid sequence set forth in SEQ ID NO: 1, has tyrosine as an amino acid corresponding to position 40 of the amino acid sequence set forth in SEQ ID NO: 1, and has a fermented milk viscosity improving effect; and
(d) a protein which is composed of an amino acid sequence encoded by a DNA that hybridizes under stringent conditions with a complementary strand of a DNA composed of the nucleotide sequence set forth in SEQ ID NO: 2, has tyrosine as an amino acid corresponding to position 40 of the amino acid sequence set forth in SEQ ID NO: 1, and has a fermented milk viscosity improving effect.

### TECHNICAL FIELD

The present invention relates to a protein having a fermented milk viscosity improving effect, fermented milk using the same, and a method for producing the same, and more particularly relates to a protein having a fermented milk viscosity improving effect, a DNA, a vector, a lactic acid bacterium, and a lactic acid bacteria composition, as well as fermented milk using these and a fermented milk thickener, and a method for producing these, a method for improving the viscosity of fermented milk, and a method for evaluating lactic acid bacteria.

### BACKGROUND ART

Fermented milk is a food that is widely eaten in general, and in Japan's “Ministerial Ordinance on Milk and Milk Products Concerning Compositional Standards, etc. (Ministerial Ordinance on Milk and Milk Products)”, fermented milk is defined as “products which are obtained by fermenting milk, or milk, etc. containing an equal or greater amount of milk solids-not-fat with lactic acid bacteria or yeast and then forming a paste or liquid, or the frozen product”. Representative examples of such fermented milk include yogurt such as set type yogurt (solid fermented milk), soft type yogurt (pasty fermented milk), and drink type yogurt (liquid fermented milk). In recent years, with the diversification of consumers' tastes, there are various demands for fermented milk, and in particular, the demand for thick fermented milk with high viscosity and viscosity is increasing.

In the production of fermented milk such as yogurt, raw material milk is mainly seeded with lactic acid bacteria and fermented to prepare fermented milk, and examples of the lactic acid bacteria used include bacteria belonging to the genus Lactobacillus and Streptococcus thermophilus. Exopolysaccharide (EPS) produced by lactic acid bacteria is known as a component involved in the viscosity of fermented milk. For example, Japanese Unexamined Patent Application Publication No. 2016-178911 (Patent literature 1) describes a method for producing a fermented soymilk product characterized by fermenting with lactic acid bacteria that produce a viscous exopolysaccharide, and examples of the lactic acid bacteria include Lactococcus lactis subsp. cremoris FC (FERM P-20185). Further, Japanese Unexamined Patent Application Publication No. 2018-143220 (Patent literature 2) describes fermented milk containing viscous polysaccharides of lactic acid bacteria and having a median diameter of 1 μm or more and 30 μm or less, and examples of the lactic acid bacteria include Streptococcus thermophilus SBT0087.

In addition, Japanese Unexamined Patent Application Publication No. 2007 -236227 (Patent literature 3) describes a composition for preventing hepatic dysfunction containing lactic acid bacteria or a culture thereof as an active ingredient, and states that syneresis or whey separation is suppressed by using Lactobacillus helveticus SR-1 (FERM P-20600) like as the lactic acid bacteria. Further, Japanese Unexamined Patent Application Publication No. Hei 7-255465 (Patent literature 4) describes Bifidobacterium longum SBT10013 as a Bifidobacterium strain characterized by extracellularly producing a large quantity of polysaccharides. Also, several studies have been made so far on proteins involved in the viscosity of fermented milk and nucleotide sequences encoding them. However, the information on the proteins involved in viscosity is still insufficient, and there is a need to provide a new protein having a fermented milk viscosity improving effect.

### CITATION LIST

### Patent Literature

[PTL 1] Japanese Unexamined Patent Application Publication No. 2016-178911

[PTL 2] Japanese Unexamined Patent Application Publication No. 2018-143220

[PTL 3] Japanese Unexamined Patent Application Publication No. 2007-236227

[PTL 4 ] Japanese Unexamined Patent Application Publication No. Hei 7-255465

### SUMMARY OF INVENTION

### Technical Problem

The present invention has been made in view of the above-mentioned object of the prior art, and aims to provide a novel protein having a fermented milk viscosity improving effect, fermented milk excellent in viscosity, and a method for producing the same.

### Solution to Problem

The present inventors have conducted intensive studies to achieve the above object, and have clarified a protein that improves the viscosity of fermented milk and a gene encoding the same. Specifically, it was known that fermented milk fermented with Lactobacillus delbrueckii subsp. bulgaricus OLL1073R-1 (accession number: FERM BP-10741) (hereinafter sometimes referred to as “R-1 strain”) tended to be more viscous than fermented milk fermented with other lactic acid bacteria strains, but it was not elucidated which gene or protein of the R-1 strain improved the viscosity of fermented milk. In view of the above, in order to elucidate a novel protein having a fermented milk viscosity improving effect, the present inventors first showed that the fermented milk fermented with the R-1 strain certainly had higher viscosity than fermented milk fermented with Lactobacillus delbruecki subsp. bulgaricus 2038 (hereinafter sometimes referred to as “2038 strain”).

Next, since exopolysaccharide (EPS) produced by lactic acid bacteria is known as a component involved in viscosity, the sequences of the EPS gene cluster thought to be involved in EPS biosynthesis were compared between the two. As a result, it was found that the R-1 strain and the 2038 strain had differences in the nucleotide sequences of the eps gene and the epsF gene. Furthermore, the epsC gene or epsF gene of the R-1 strain was introduced into the 2038 strain to prepare a transformant, and only the transformant introduced with the epsC gene of the strain R-1 had improved viscosity. Therefore, it has been found that the protein encoded by the epsC gene of the R-1 strain has a fermented milk viscosity improving effect.

In addition, on Feb. 5, 2020, the nucleotide sequence of R-1 strain epsC was used as a query to perform a web Blast (parameters: default values) on the NCBI nt database, and the sequence of the top hit was the epsC gene of the 2038 strain, with a Query Cover of 100% and a Per. Ident of 99.87%. Furthermore, the base difference was also reflected in the amino acid difference. Therefore, the present inventors also found that the nucleotide sequence of the epsc gene of the R-1 strain and the protein that is a product thereof are novel. Thus, the present invention has been completed.

Specifically, the present invention relates to a protein having a fermented milk viscosity improving effect, fermented milk using the same, a method for producing the same, and the like, and more specifically, it is as follows.

[1]

At least one protein selected from the group consisting of the following proteins (a) to (d):
(a) a protein which is composed of an amino acid sequence set forth in SEQ ID NO: 1;
(b) a protein which is, in the amino acid sequence set forth in SEQ ID NO: 1, composed of an amino acid sequence in which one or more amino acids other than tyrosine at position 40 are substituted, deleted, inserted, and/or added and has a fermented milk viscosity improving effect;
(c) a protein which is composed of an amino acid sequence having 80% or more identity with the amino acid sequence set forth in SEQ ID NO: 1, has tyrosine as an amino acid corresponding to position 40 of the amino acid sequence set forth in SEQ ID NO: 1, and has a fermented milk viscosity improving effect; and
(d) a protein which is composed of an amino acid sequence encoded by a DNA that hybridizes under stringent conditions with a complementary strand of a DNA composed of the nucleotide sequence set forth in SEQ ID NO: 2, has tyrosine as an amino acid corresponding to position 40 of the amino acid sequence set forth in SEQ ID NO: 1, and has a fermented milk viscosity improving effect.
   [1′]

A composition comprising: at least one selected from the group consisting of the proteins (a) to (d) (preferably a composition for use in improving the viscosity of fermented milk).

[2]

A DNA encoding the protein according to [1].

[2′]

A composition comprising: at least one selected from the group consisting of DNAs encoding the proteins (a) to (d) (preferably a composition for use in improving the viscosity of fermented milk).

[3]

A vector comprising: the DNA according to [2].

[3′]

A vector comprising: at least one selected from the group consisting of DNAs encoding the proteins (a) to (d).

[4]

A composition comprising: at least one selected from the group consisting of the protein according to [1], the DNA according to [2], and the vector according to [3].

[5]

A lactic acid bacterium introduced with at least one selected from the group consisting of the DNA according to [2] and the vector according to [3].

[5′]

A lactic acid bacterium introduced with the vector according to [3′] (preferably a lactic acid bacterium having a fermented milk viscosity improving effect).

[6]

A lactic acid bacterium comprising: the DNA according to [2].

[7]

The lactic acid bacterium according to [6], which has a fermented milk viscosity improving effect.

[8]

A lactic acid bacteria composition comprising : the lactic acid bacterium according to any one of [5] to [7].

[8′]

A lactic acid bacteria composition comprising: the lactic acid bacterium according to [5′] (preferably a lactic acid bacteria composition for use in improving the viscosity of fermented milk).

[9]

The lactic acid bacteria composition according to [8] or [8′], which is fermented milk.

[10]

The lactic acid bacteria composition according to [8], [8], or [9], further comprising; an exopolysaccharide derived from the lactic acid bacterium according to any one of [5] to [7] or [5′].

[11]

A method for producing fermented milk, comprising: a fermentation step of adding the lactic acid bacterium according to any one of [5] to [7] or [5′] or the lactic acid bacteria composition according to any one of [8] to [10] or [8] to a milk preparation solution containing raw material milk and fermenting the mixture.

[12]

A method for improving the viscosity of fermented milk, comprising: a fermentation step of adding the lactic acid bacterium according to any one of [5] to [7] or [5′] or the lactic acid bacteria composition according to any one of [8] to or [8′] to a milk preparation solution containing raw material milk and fermenting the mixture.

A method for evaluating lactic acid bacteria for presence or absence of a fermented milk viscosity improving effect using as an index at least one selected from the group consisting of DNAs encoding the following proteins (a) to (d);
(a) a protein which is composed of an amino acid sequence set forth in SEQ ID NO: 1;
(b) a protein which is, in the amino acid sequence set forth in SEQ ID NO: 1, composed of an amino acid sequence in which one or more amino acids other than tyrosine at position 40 are substituted, deleted, inserted, and/or added and has a fermented milk viscosity improving effect;
(c) a protein which is composed of an amino acid sequence having 80% or more identity with the amino acid sequence set forth in SEQ ID NO: 1, has tyrosine as an amino acid corresponding to position 40 of the amino acid sequence set forth in SEQ ID NO: 1, and has a fermented milk viscosity improving effect; and
(d) a protein which is composed of an amino acid sequence encoded by a DNA that hybridizes under stringent conditions with a complementary strand of a DNA composed of the nucleotide sequence set forth in SEQ ID NO: 2, has tyrosine as an amino acid corresponding to position 40 of the amino acid sequence set forth in SEQ ID NO: 1, and has a fermented milk viscosity improving effect.
   [14]

Fermented milk comprising: a lactic acid bacterium evaluated as having a fermented milk viscosity improving effect by the method for evaluating lactic acid bacteria according to [13].

[15]

A method for producing lactic acid bacteria, comprising:
an evaluation step of evaluating whether or not the lactic acid bacteria have a fermented milk viscosity improving effect by the method for evaluating lactic acid bacteria according to; and
a step of obtaining lactic acid bacteria evaluated as having a fermented milk viscosity improving effect in the evaluation step.
   [16]

A method for producing fermented milk, comprising:
an evaluation step of evaluating whether or not the lactic acid bacteria have a fermented milk viscosity improving effect by the method for evaluating lactic acid bacteria according to; and
a fermentation step of adding the lactic acid bacteria evaluated as having a fermented milk viscosity improving effect in the evaluation step to a milk preparation solution containing raw material milk and fermenting the mixture.
   [17]

A method for improving the viscosity of fermented milk, comprising:
an evaluation step of evaluating whether or not the lactic acid bacteria have a fermented milk viscosity improving effect by the method for evaluating lactic acid bacteria according to [13]; and
a fermentation step of adding the lactic acid bacteria evaluated as having a fermented milk viscosity improving effect in the evaluation step to a milk preparation solution containing raw material milk and fermenting the mixture.
   [18]

A fermented milk thickener comprising: an exopolysaccharide derived from the lactic acid bacterium according to any one of [5] to [7] or [5′] as an active ingredient.

[19]

A method for producing an exopolysaccharide of lactic acid bacteria, comprising: a step of adding the lactic acid bacterium according to any one of [5] to [7] or [5′] or the lactic acid bacteria composition according to any one of [8] to [10] or [8′] to a medium containing glucose and/or a saccharide composed of glucose, fermenting the mixture, and collecting an exopolysaccharide contained in a fermented product.

[20]

A method for producing an exopolysaccharide of lactic acid bacteria, comprising:
an evaluation step of evaluating whether or not the lactic acid bacteria have a fermented milk viscosity improving effect by the method for evaluating lactic acid bacteria according to [13]; and
a step of adding the lactic acid bacteria evaluated as having a fermented milk viscosity improving effect in the evaluation step to a medium containing glucose and/or a saccharide composed of glucose, fermenting the mixture, and collecting an exopolysaccharide contained in a fermented product.
   [21]

A method for producing a fermented milk thickener, comprising:
a fermentation step of adding the lactic acid bacterium according to any one of [5] to [7] or [5′] or the lactic acid bacteria composition according to any one of [8] to [10] or [8′] to a medium containing glucose and/or a saccharide composed of glucose, fermenting the mixture, and obtaining a fermented product containing an exopolysaccharide; and
a step of obtaining a fermented milk thickener containing the exopolysaccharide as an active ingredient.
   [22]

A method for producing a fermented milk thickener, comprising:
an evaluation step of evaluating whether or not the lactic acid bacteria have a fermented milk viscosity improving effect by the method for evaluating lactic acid bacteria according to [13];
a fermentation step of adding the lactic acid bacteria evaluated as having a fermented milk viscosity improving effect in the evaluation step to a medium containing glucose and/or a saccharide composed of glucose, fermenting the mixture, and obtaining a fermented product containing an exopolysaccharide; and
a step of obtaining a fermented milk thickener containing the exopolysaccharide as an active ingredient.

### Advantageous Effects of Invention

The present invention makes it possible to provide a novel protein having a fermented milk viscosity improving effect, fermented milk excellent in viscosity, and a method for producing the same. More specifically, it becomes possible to provide a novel protein having a fermented milk viscosity improving effect, a DNA encoding the protein, a vector containing the DNA, a lactic acid bacterium containing the DNA or the vector, and a lactic acid bacteria composition thereof, as well as fermented milk excellent in viscosity and a fermented milk thickener, and a method for producing them, a method for improving the viscosity of fermented milk, and a method for evaluating lactic acid bacteria.

For example, by introducing a DNA encoding the novel protein of the present invention into various lactic acid bacteria, it becomes possible to easily produce fermented milk with higher viscosity and thickness than conventional ones. In addition, by using the sequence of a DNA encoding the novel protein of the present invention as a selection criterion, it becomes possible to easily select lactic acid bacteria capable of producing fermented milk with higher viscosity and thickness than conventional ones. Furthermore, the novel protein of the present invention can improve the viscosity of fermented milk, thereby reducing the amount of free whey and improving the appearance of the fermented milk.

### BRIEF DESCRIPTION OF DRAWINGS

FIG. 1 is a graph showing the adhesion time (seconds) of the fermented milk fermented with the 2038 strain or the R-1 strain, which was obtained in Adhesion Time Measurement of <Viscosity Evaluation 1>.

FIG. 2 is a schematic diagram of EPS gene cluster 1 and EPS gene cluster 2 in <Comparison of EPS Gene Cluster Regions>.

FIG. 3 is a photograph showing the appearance of the fermented milk fermented with 2038 strain, R-1 strain, 2038-epsC strain, or 2038-epsF strain, which was obtained in Preparation of Fermented Milk of <Viscosity Evaluation 2>.

FIG. 4 is a graph showing the adhesion time (seconds) of the fermented milk fermented with the 2038 strain, R-1 strain, 2038-epsC strain, or 2038 -eps strain, which was obtained in Adhesion Time Measurement of <Viscosity Evaluation 2>.

### DESCRIPTION OF EMBODIMENTS

Hereinafter, the present invention will be described in detail with reference to preferred embodiments thereof.

<Protein, DNA, Vector, and Composition Containing the Same>

The protein of the present invention is a protein having a fermented milk viscosity improving effect and is at least one protein selected from the group consisting of the following proteins (a) to (d):
(a) a protein which is composed of an amino acid sequence set forth in SEQ ID NO: 1;
(b) a protein which is, in the amino acid sequence set forth in SEQ ID NO: 1, composed of an amino acid sequence in which one or more amino acids other than tyrosine at position 40 are substituted, deleted, inserted, and/or added and has a fermented milk viscosity improving effect;
(c) a protein which is composed of an amino acid sequence having 80% or more identity with the amino acid sequence set forth in SEQ ID NO: 1, has tyrosine as an amino acid corresponding to position 40 of the amino acid sequence set forth in SEQ ID NO: 1, and has a fermented milk viscosity improving effect; and
(d) a protein which is composed of an amino acid sequence encoded by a DNA that hybridizes under stringent conditions with a complementary strand of a DNA composed of the nucleotide sequence set forth in SEQ ID NO: 2, has tyrosine as an amino acid corresponding to position 40 of the amino acid sequence set forth in SEQ ID NO: 1, and has a fermented milk viscosity improving effect.

The protein of the present invention is a protein that has a fermented milk viscosity improving effect (hereinafter sometimes referred to as “viscosity improving protein”). In the present invention, the “fermented milk viscosity” indicates the property of drawing strings due to the viscosity and/or elasticity of fermented milk, and the “fermented milk viscosity improving effect” indicates the action of imparting the above viscosity to fermented milk or improving the above viscosity of fermented milk (in the present specification, sometimes referred to as “viscosity improving effect”). The reason why the protein of the present invention has the above viscosity improving effect is unclear, but the present inventors presume that this is because the protein of the present invention acts in the process of EPS biosynthesis by lactic acid bacteria to produce EPS with high viscosity structure.

In the present invention, the viscosity of fermented milk can be evaluated, for example, by the time (adhesion time) during which the fermented milk maintains a filamentous unit when stretched. In the present invention, for example, the adhesion time can be measured using a creepmeter (model: RE2-33005S (manufactured by Yamaden Co., Ltd.), container: a cylindrical container with φ=41 mm and height=35 mm, jig: φ=25.2 mm, height=25 mm, rate: 10 mm/sec, return distance: 10 mm, amount of fermented milk: 10 g) as the time required for the fermented milk adhering to the jig to be completely separated from the fermented milk in the container after the jig is lifted, where the moment immediately after the fermented milk is compressed twice is taken as 0 seconds. It can be evaluated that the longer the adhesion time, the higher the viscosity of the fermented milk.

The DNA of the present invention is a DNA encoding the above viscosity improving protein (hereinafter sometimes referred to as “viscosity improving DNA”). Specifically, the DNA of the present invention is at least one DNA selected from the group consisting of the following DNAs (a′) to (d′).
(a′) a DNA encoding a protein which is composed of an amino acid sequence set forth in SEQ ID NO: 1;
(b′) a DNA encoding a protein which is, in the amino acid sequence set forth in SEQ ID NO: 1, composed of an amino acid sequence in which one or more amino acids other than tyrosine at position 40 are substituted, deleted, inserted, and/or added and has a fermented milk viscosity improving effect;
(c′) a DNA encoding a protein which is composed of an amino acid sequence having 80% or more identity with the amino acid sequence set forth in SEQ ID NO: 1, has tyrosine as an amino acid corresponding to position 40 of the amino acid sequence set forth in SEQ ID NO: 1, and has a fermented milk viscosity improving effect; and
(d′) a DNA encoding a protein which is composed of an amino acid sequence encoded by a DNA that hybridizes under stringent conditions with a complementary strand of a DNA composed of the nucleotide sequence set forth in SEQ ID NO: 2, has tyrosine as an amino acid corresponding to position 40 of the amino acid sequence set forth in SEQ ID NO: 1, and has a fermented milk viscosity improving effect.

The “ (a) amino acid sequence set forth in SEQ ID NO: 1” is an amino acid sequence encoded by the epsc gene of Lactobacillus delbrueckii subsp. bulgaricus OLL1073R-1 (accession number: FERM BP-10741) (R-1 strain). The “ (a′) DNA encoding an amino acid sequence set forth in SEQ ID NO: 1” is not particularly limited as long as it encodes the amino acid sequence, but is preferably the nucleotide sequence set forth in SEQ ID NO: 2. The nucleotide sequence set forth in SEQ ID NO: 2 is the nucleotide sequence of the epsC gene of the R-1 strain. As described above, the present inventors have found that the protein encoded by the epsC gene of the R-1 strain has a fermented milk viscosity improving effect. In the amino acid sequence set forth in SEQ ID NO: 1, it is particularly important that the amino acid at position 40 is tyrosine. If such amino acid is substituted with a different amino acid (for example, 2038 strain or 2038-epsF strain in Examples), excellent viscosity is not exhibited in fermented milk even if other sequences are common. Hereinafter, the amino acid sequence set forth in SEQ ID NO: 1 is sometimes referred to as “R1-EpsC”, and the nucleotide sequence set forth in SEQ ID NO: 2 is sometimes referred to as “R1-epsC”.

In addition, in the natural world, mutations in nucleotide sequences can cause mutations in the amino acid sequences of proteins encoded by those sequences. Furthermore, in the current state of the art, for example, when the nucleotide sequence information of the epsC gene of the R-1 strain (R1-epsC) or the amino acid sequence information of the protein encoded by it (R1-EpsC) is obtained, those skilled in the art can also modify the nucleotide sequence to prepare a viscosity improving protein with an amino acid sequence different from the one encoded but with a maintained or improved viscosity improving effect.

Therefore, another aspect of the “viscosity improving protein” according to the present invention also includes “(b) a protein which is, in the amino acid sequence set forth in SEQ ID NO: 1, composed of an amino acid sequence in which one or more amino acids other than tyrosine at position 40 are substituted, deleted, inserted, and/or added and has a fermented milk viscosity improving effect”. Further, another aspect of the “viscosity improving DNA” according to the present invention also includes “ (b′) a DNA encoding a protein which is, in the amino acid sequence set forth in SEQ ID NO: 1, composed of an amino acid sequence in which one or more amino acids other than tyrosine at position 40 are substituted, deleted, inserted, and/or added and has a fermented milk viscosity improving effect”. Here, “more” refers to the number of amino acid modifications within the scope where the protein (variant) after substitution, deletion, insertion, and/or addition (hereinafter collectively referred to as a “modification” in some cases) has a viscosity improving effect, and is usually 100 or less, 1 to 80, preferably 1 to 40, more preferably 1 to 20, and further preferably 1 to several (for example, 1 to 10, 1 to 8, 1 to 4, and 1 to 2).

For example, based on the nucleotide sequence information of the epsC gene of the R-1 strain (R1-epsC), those skilled in the art can prepare a polynucleotide encoding such a variant using a known site-directed mutagenesis method or the like.

Furthermore, in the current state of the art, when the nucleotide sequence information of the epsC gene of the R-1 strain (R1-epsC) is obtained, those skilled in the art can obtain polynucleotides (homologous genes) encoding viscosity improving proteins from microorganisms other than the R-1 strain by hybridization technique (Southern, E. M., J. Mol. Biol. , 98: 503, 1975), polymerase chain reaction (PCR) technique (Saiki, R. K., et al. Science, 230: 1350-1354, 1985, Saiki, R. K. et al. Science, 239: 487-491, 1988), and the like. Therefore, the aspect of the “viscosity improving protein” according to the present invention also includes “(d) a protein which is composed of an amino acid sequence encoded by a DNA that hybridizes under stringent conditions with a complementary strand of a DNA composed of the nucleotide sequence set forth in SEQ ID NO: 2, has tyrosine as an amino acid corresponding to position 40 of the amino acid sequence set forth in SEQ ID NO: 1, and has a fermented milk viscosity improving effect”. In addition, another aspect of the “viscosity improving DNA” according to the present invention also includes “(d′) a DNA encoding a protein which is composed of an amino acid sequence encoded by a DNA that hybridizes under stringent conditions with a complementary strand of a DNA composed of the nucleotide sequence set forth in SEQ ID NO: 2, has tyrosine as an amino acid corresponding to position 40 of the amino acid sequence set forth in SEQ ID NO: 1, and has a fermented milk viscosity improving effect ”. Note that in the present invention, the “amino acid corresponding to position 40 of the amino acid sequence set forth in SEQ ID NO: 1” refers to the amino acid at the position aligned with tyrosine at position 40 in R1-EpsC when arranged with the amino acid sequence set forth in SEQ ID NO: 1 (R1-EpsC) using nucleotide sequence and amino acid sequence analysis software (such as GENETYX-MAC or Sequencher), BLAST (Basic Local Alignment Search Tool at the National Center for Biological Information), or the like (for example, parameters: default values (that is, initially set values)).

Hybridization reactions are usually performed under stringent conditions to isolate homologous genes. The “stringent conditions” means that the washing operation on the membrane after hybridization is performed in a high-temperature, low-salt solution, and is, for example, conditions of washing in 0.5% SDS solution at. 60° C. for 20 minutes at 2× SSC concentration (1×SSC: 15 mM trisodium citrate, 150 mM sodium chloride). In addition, hybridization can be performed, for example, according to the method described in the instruction manual attached to the known ECL Direct DNA/RNA Labeling/Detection System (manufactured by Amersham Pharmacia Biotech). The more stringent the hybridization conditions, the more likely it is to isolate DNA of high identity. However, the above conditions are only examples, and the necessary stringency can be achieved by appropriately combining DNA concentration, DNA length, hybridization reaction time, and the like.

Furthermore, the protein encoded by the homologous gene obtained by such methods usually has a high homology with the amino acid sequence set forth in SEQ ID NO: 1 (R1-EpsC). Therefore, the aspect of the “viscosity improving protein” according to the present invention also includes “(c) a protein which is composed of an amino acid sequence having 80% or more identity with the amino acid sequence set forth in SEQ ID NO: 1, has tyrosine as an amino acid corresponding to position 40 of the amino acid sequence set forth in SEQ ID NO: 1, and has a fermented milk viscosity improving effect”. In addition, the aspect of the “viscosity improving DNA” according to the present invention also includes “(c′) a DNA encoding a protein which is composed of an amino acid sequence having 80% or more identity with the amino acid sequence set forth in SEQ ID NO: 1, has tyrosine as an amino acid corresponding to position 40 of the amino acid sequence set forth in SEQ ID NO: 1, and has a fermented milk viscosity improving effect”.

The identity of amino acid sequences can be determined, for example, using the above BLAST or the like (for example, parameters: default values (that is, initially set values)). The identity with the amino acid sequence set forth in SEQ ID NO: 2 (R1-EpsC) is usually 80% or more, preferably 90% or more, and more preferably 95% or more (for example, 96% or more, 97% or more, 98% or more, and 99% or more).

The viscosity improving protein encoded by a homologous gene may be a protein encoded by a gene isolated from a microorganism different from lactic acid bacteria, but is preferably isolated from lactic acid bacteria. Examples of the lactic acid bacteria include the family Streptococcaceae, the family Lactobacillus, and the family Leuconostocaceae, and more specific examples include lactic acid bacilli such as the genus Lactobacillus, the genus Lacticaseibacillus, the genus Lactiplantibacillus, the genus Liquorilactobacillus, the genus Limosilactobacillus, the genus Levilactobacillus, the genus Lentilactobacillus, and the genus Weissella; lactic acid cocci such as the genus Pediococcus, the genus Leuconostoc, the genus Lactococcus, the genus Streptococcus, and the genus Enterococcus; and the genus Bifidobacterium. Among these, the genus Lactobacillus is preferable, Lactobacillus delbrueckii (including subspecies) is more preferable, and Lactobacillus delbrueckii subspecies bulgaricus is further preferable.

In the present invention, whether each protein has the above viscosity improving effect can be confirmed as follows, for example. A DNA encoding each protein or a vector containing the DNA is expressibly introduced into at least one lactic acid bacterium selected from the group consisting of Lactobacillus delbrueckii, delbrueckii subspecies preferably Lactobacillus bulgaricus (but not having a DNA encoding any of the proteins (a) to (d), that is, not having any of the DNAs (a′) to (d′)). The adhesion time of fermented milk obtained under the same fermentation conditions (for example, conditions that allow fermentation of the lactic acid bacterium before introduction) using the lactic acid bacterium before the introduction is set to 1. Then, the adhesion time of the fermented milk obtained using the introduced lactic acid bacterium (transformant) is 2 or more, preferably 3 or more, and more preferably 4 or more. Specifically, the lactic acid bacterium introduced with the DNA or vector is preferably, for example, Lactobacillus delbrueckii subspecies bulgaricus 2038 strain (2038 strain). The 2038 strain can be isolated by smearing a diluted solution of Meiji Bulgaria Yogurt LB81 (manufactured by Meiji Co., Ltd.) on a BCP agar medium, culturing at 37° C. for 48 hours, and then picking up rough colonies.

The adhesion time can be measured using the above creepmeter under the above conditions as the time required for the fermented milk adhering to the jig to be completely separated from the fermented milk in the container after the jig is lifted, where the moment immediately after each fermented milk is compressed twice is taken as 0 seconds. In addition, whether each lactic acid bacterium does not have any of the above DNAs (a′) to (d′) can be appropriately confirmed by a known method or a method according thereto based on the nucleotide sequences of these DNAs. For example, it can be confirmed by the method for detecting viscosity improving DNA described in the Evaluation Step of <Method for Evaluating Lactic Acid Bacteria> to be described later. Furthermore, as a method for introducing the DNA or vector encoding the protein into the lactic acid bacterium, a known method or a method according thereto can be appropriately selected. For example, in the method described in [Viscosity Improving Protein] below, the above lactic acid bacterium can be used as a host cell.

### Viscosity Improving Protein

The viscosity improving protein according to the present invention can be obtained by appropriately using a known method or a method according thereto. It can be obtained, for example, by a production method including the steps of culturing a host cell introduced with at least one selected from the group consisting of a DNA encoding the viscosity improving protein and a vector containing the DNA, and collecting the protein expressed in the host cell. More specifically, first, the conventional method is used to obtain a DNA encoding the viscosity improving protein (viscosity improving DNA) as an isolated DNA from the target microorganism having at least one of the above DNAs (a′) to (d′), such as the R-1 strain. The isolated DNA may be a chemically synthesized DNA obtained by artificially chemically synthesizing the viscosity improving DNA. Next, DNA (the above isolated DNA) or an expression containing the same is prepared, which is introduced into host cells to culture transformants. This allows the transformant to express the viscosity improving protein of the present invention, making it possible to obtain this protein as a recombinant protein from the culture.

Examples of methods for obtaining the above isolated DNA from the target microorganism include a method in which genomic DNA extracted from the above microorganism or cDNA synthesized based on MRNA extracted from the above microorganism is ligated with a vector such as plasmid vector, phage vector, cosmid vector, BAC vector, or PAC vector to prepare a DNA library or cDNA library, and the desired genomic DNA or cDNA is isolated from the library by hybridization using a probe made based on the nucleotide sequence of the viscosity improving DNA (such as R1-epsC) ; and a method in which a primer prepared based on the nucleotide sequence of the viscosity improving DNA (such as R1-epsC) is used to perform PCR using the genomic DNA of the target microorganism or the above cDNA as a template, and the amplified DNA fragment is ligated as necessary with an appropriate vector to isolate the desired genomic DNA.

The expression vector is a vector that is replicable in a host cell and contains a protein encoded by its polynucleotide sequence in a state expressible in the host cell. Such expression vectors can be constructed, for example, based on autonomously replicating vectors, that is, ones which exist as extrachromosomal entities and whose replication does not depend on chromosomal replication, for example plasmids. In addition, the above expression vector may be constructed based on phage DNA which is, when introduced into a host cell, integrated into the genome of the host cell and replicates together with the chromosome into which it has been integrated. Examples of the plasmid include Escherichia coli-derived plasmids (such as pET22, pBR322, pBR325, pUC118, pUC119, pUC18, and pUC19), yeast-derived plasmids (such as YEp13, YEp24, and YCp50), Bacillus subtilis-derived plasmids (such as pUB110 and pTP5), and shuttle vectors between Escherichia coli and lactic acid bacteria (such as pGMβ1). Examples of the phage DNA include λ phage (such as Charon4A, Charon21A, EMBL3, EMBL4, λgt10, λgt11, and λZAP).

As the procedure and method for constructing the expression vector, a known method or a method according thereto can be appropriately employed. For example, in order to insert the viscosity improving DNA into a vector, a method or the like is employed in which the isolated DNA is first cleaved with an appropriate restriction enzyme, inserted into the restriction enzyme site or multicloning site of an appropriate plasmid, and ligated to the plasmid.

In order to be actually introduced into host cells to express the viscosity improving protein, the expression vector preferably contains, in addition to the DNA encoding the viscosity improving protein of the present invention (viscosity improving DNA), a polynucleotide sequence that controls its expression, a polynucleotide sequence that induces expression other than the polynucleotide sequence that controls expression, a genetic marker for selecting cells, and the like.

Examples of the polynucleotide sequence that controls expression include polynucleotide sequences encoding promoters, terminators, and signal peptides, which may be one or a combination of two or more among them. The promoter is not particularly limited as long as it exhibits transcriptional activity in the host cell, and may be a polynucleotide sequence that controls the expression of a gene encoding a protein homologous or heterologous to the host cell. If the host cell is a bacterium, examples of the polynucleotide sequence that controls expression include the lactose operon, which can induce expression of genes located downstream by the addition of isopropyl-β-D-thiogalactopyranoside (IPTG). The gene marker can be appropriately selected according to the method for selecting transformants. For example, a gene encoding drug resistance or a gene complementing auxotrophy can be used.

The host cell is not particularly limited, but is preferably a microorganism. and examples thereof include filamentous fungi, yeasts, Escherichia coli, actinomycetes, and lactic acid bacteria. When used in the method for producing the viscosity improving protein of the present invention, the host cell is not particularly limited, but is preferably a lactic acid bacterium, if the host cell introduced with the DNA is used as it is for <Method for Producing Fermented Milk>, <Method for Improving Viscosity of Fermented Milk>, <Method for Producing Exopolysaccharide of Lactic Acid Bacteria>, or <Method for Producing fermented milk thickener> to be described later. The host cells may be those already transformed to delete specific functions, or mutants, if necessary.

As a method for introducing the DNA or expression vector into these host cells, a known method or a method according thereto can be appropriately employed, and examples thereof include the shock method, electroporation method, spheroplast method, and lithium acetate method, and methods for introduction into lactic acid bacteria include the conjugation method. In addition, methods for introduction into plant cells include methods using Agrobacterium and the particle gun method, methods for introduction into insect cells include methods using baculovirus and the electroporation method, and methods for introduction into animal cells include the calcium phosphate method, lipofection method, and electroporation method.

By culturing the transformant having the DNA or expression vector introduced into the host cell in this way in an appropriate medium, the viscosity improving protein of the present invention can be collected from the culture thereof (for example, cultured microbial cells). Therefore, the present invention can also provide a method for producing the viscosity improving protein of the present invention, which includes the steps of culturing the transformant and collecting the viscosity improving protein expressed in the transformant.

As the culture conditions for the transformant, for example, the culture conditions for host cells can be applied. Those skilled in the art can appropriately adjust and set the temperature, presence/absence of air addition, oxygen concentration, carbon dioxide concentration, medium pH, culture temperature, culture time, humidity, and the like, according to the type of host cell, the medium used, and the like. In addition, as a method for collecting the viscosity improving protein from the culture, for example, it is also possible to use a method in which the viscosity improving protein is expressed in a host cell (for example, Escherichia coli), and after culturing the transformant, the cultured cells are collected by centrifugation, filtration, or the like, and the liquid obtained by crushing the cells is obtained as a crudely purified product. Furthermore, this supernatant can be concentrated by ultrafiltration or the like, and a preservative or the like can be added to obtain a concentrated crudely purified product. Further, the crudely purified product or the concentrated crudely purified product may be purified by, for example, salting-out method, organic solvent precipitation method, membrane separation method, or chromatographic separation method, either alone or in combination of two or more thereof. Alternatively, purification may be performed by expressing a viscosity improving protein tagged for purification in a host cell (for example, Escherichia coli), passing the crude extract through a tagged protein purification column, and then eluting the tagged protein.

Other compounds may be added directly or indirectly to the viscosity improving protein of the present invention. Such addition is not particularly limited, and may be addition at the gene level or chemical addition. Also, the addition site is not particularly limited either, and may be either the amino terminus (hereinafter also referred to as “N terminus”) or the carboxy terminus (hereinafter also referred to as “C terminus”) of the viscosity improving protein of the present invention, or both. Addition at the gene level is achieved by using a DNA encoding the viscosity improving protein of the present invention (viscosity improving DNA), to which a DNA encoding another protein is added in the same reading frame. The “another protein” added in this way is not particularly limited. For example, for the purpose of facilitating purification of the viscosity improving protein of the present invention, purification tag proteins are preferably used, such as polyhistidine (His-) tagged (tag) protein, FLAG-tagged protein (registered trademark, Sigma-Aldrich), and glutathione S-transferase (GST). For example, for the purpose of facilitating the detection of the viscosity improving protein 4 the present invention, detection tag proteins are preferably used, such as fluorescent proteins such as GFP and chemiluminescent proteins such as luciferase. Chemical addition may be covalent bond or non-covalent bond. The “covalent bond” is not particularly limited, and examples thereof include an amide bond between an amino group and a carboxy group, an alkylamine bond between an amino group and an alkyl halide group, a disulfide bond between thiols, and a thioether bond between a thiol group and a maleimide group or an alkyl halide group. Examples of the “non-covalent bond” include a biotin-avidin bond.

### Viscosity Improving DNA

As long as the viscosity improving DNA of the present invention encodes the amino acid sequence of the viscosity protein improving of the present invention, it may be a DNA obtained by introducing a mutation into a natural DNA, may be a DNA composed of an artificially designed nucleotide sequence, or may be partially or wholly composed of non-natural nucleotides. Furthermore, the form thereof is not particularly limited, and examples thereof include cDNA, genomic DNA, and chemically synthesized DNA, which are exemplified as isolated DNAs in [Viscosity—Improving Protein] described above.

Furthermore, from the viewpoint of further improving the expression efficiency of the encoded viscosity improving protein in a host cell, the viscosity improving DNA of the present invention can also take the form of a DNA encoding the viscosity improving protein of the present invention having codons optimized according to the type of the host cell.

### Vector

The viscosity improving DNA of the present invention can also take the form of a vector into which that DNA is inserted so that the DNA can be replicated in the host cell. Accordingly, the present invention also provides a vector including the viscosity improving DNA of the present invention. Examples of the vector of the present invention include the expression vectors exemplified in [Viscosity Improving Protein] described above, including preferable embodiments thereof.

### Composition

The present invention provides a composition including at least one of the above-described viscosity improving protein, viscosity improving DNA, and vector of the present invention. The composition of the present invention can be a composition for use in improving the viscosity of fermented milk, including at least one of the viscosity improving protein, viscosity improving DNA, and vector of the present invention as an active ingredient. For example, by introducing the composition of the present invention into various lactic acid bacteria to form the following lactic acid bacteria of the present invention and using them to produce fermented milk, it is possible to obtain fermented milk with higher viscosity and thickness than conventional ones.

The composition of the present invention may further contain additional components. Examples of the additional components include, but are not limited to, sterile water, physiological saline, vegetable oils, surfactants, lipids, solubilizers, buffers, DNase inhibitors, and preservatives, and one of these may be used alone, or a combination of two or more thereof may be used.

### Lactic Acid Bacteria and Lactic Acid Bacteria Composition

The present invention also provides a transformant in which the above-described viscosity improving DNA of the present invention or the vector of the present invention containing the viscosity improving DNA is introduced into the host cell. Examples of the transformant include the transformants exemplified in [Viscosity Improving Protein] described above.

In the present invention, the host cell for the transformant is preferably lactic acid bacteria. The “lactic acid bacteria of the present invention” include lactic acid bacteria into which at least one selected from the group consisting of the above-described viscosity improving DNA of the present invention and the vector of the present invention containing the viscosity improving DNA has been introduced; and lactic acid bacteria having the above-described viscosity improving DNA of the present invention. Furthermore, the “lactic acid bacteria of the present invention” also include lactic acid bacteria into which the viscosity improving protein of the present invention itself has been introduced. Therefore, the lactic acid bacteria of the present invention can exhibit a fermented milk viscosity improving effect.

Moreover, the lactic acid bacteria of the present invention may be in the form of a lactic acid bacteria composition, and the present invention also provides a lactic acid bacteria composition containing at least one of these lactic acid bacteria of the present invention. The lactic acid bacteria composition may be a lactic acid bacteria composition for, in addition to the use in producing the viscosity improving protein, use in producing fermented milk with improved viscosity, improving the viscosity of fermented milk, producing exopolysaccharides of lactic acid bacteria, or producing a fermented milk thickener.

### Lactic Acid Bacteria

Lactic acid bacteria as host cells into which the viscosity improving protein, viscosity improving DNA, ox vector of the present invention is introduced are not particularly limited. Examples of the lactic acid bacteria include, but are not limited to, the family Streptococcaceae, the family Lactobacillus, and the family Leuconostocaceae, and more specific examples include lactic acid bacilli such as the genus Lactobacillus, the genus Lacticaseibacillus, the genus Lactiplantibacillus, the genus Liquorilactobacillus, the genus Limosilactobacillus, the genus Levilactobacillus, the genus Lentilactobacillus, and the genus Weissella; lactic acid cocci such as the genus Pediococcus, the genus Leuconostoc, the genus Lactococcus, the genus Streptococcus, and the genus Enterococcus; and the genus Bifidobacterium. Among these, the genus Lactobacillus is preferable, Lactobacillus delbrueckii (including subspecies) is more preferable, and Lactobacillus delbrueckii subspecies bulgaricus is further preferable. The lactic acid bacteria as the host cells may already have at least one of the proteins (a) to (d) or at least one of the DNAs (a′) to (d′). When such lactic acid bacteria are used as host cells, a further viscosity improving effect can be expected.

As the method for introducing the above-described viscosity improving protein, viscosity improving DNA, or vector into these lactic acid bacteria, the method exemplified as the method for introducing DNA or an expression vector in [Viscosity Improving Protein] described above can be appropriately employed. For example, it is preferable to introduce the above-described viscosity improving DNA or vector using at least one selected from the group consisting of the heat shock method, electroporation method, spheroplast method, lithium acetate method, and bonding method.

In addition, as the lactic acid bacteria of the present invention, examples of lactic acid bacteria having the above-described viscosity improving DNA of the present invention include lactic acid bacteria having at least one of the DNAS (a′) to (d′) among the lactic acid bacteria exemplified as the host cells.

Note that whether the viscosity improving protein or viscosity improving DNA of the present invention is possessed by (preferably introduced into) the lactic acid bacteria of the present invention can be appropriately confirmed by a known method or a method according thereto. For example, it can be confirmed by the method for detecting viscosity improving DNA described in the Evaluation Step of <Method for Evaluating Lactic Acid Bacteria> to be described later. Therefore, the “lactic acid bacteria of the present invention” also include lactic acid bacteria evaluated as having a fermented milk viscosity improving effect by the method for evaluating lactic acid bacteria of the present invention to be described later (including those evaluated to have a high possibility of having the above effect), and lactic acid bacteria obtained by the method for producing lactic acid bacteria of the present invention.

The DNA possessed by (preferably introduced into) the lactic acid bacteria of the present invention may be retained in the genomic DNA of the lactic acid bacteria, and in the case of a vector, it may be replicated and retained as an independent entity outside the genomic DNA. The DNA introduced into lactic acid bacteria may be retained by being randomly inserted into the genomic DNA, or may be retained by homologous recombination. In addition, as long as the lactic acid bacterium of the present invention has a fermented milk viscosity improving effect, it may be an artificial mutant strain, natural mutant strain, or genetically modified strain of the same strain of the lactic acid bacterium or a subcultured strain thereof.

The lactic acid bacterium of the present invention preferably has a fermented milk viscosity improving effect. In the present invention, whether the lactic acid bacteria into which the viscosity improving protein, the viscosity improving DNA, or the vector has been introduced has fermented milk viscosity improving effect can be confirmed as follows, for example. The adhesion time of fermented milk obtained under the same fermentation conditions (for example, conditions that allow fermentation of the lactic acid bacterium before introduction) using the lactic acid bacterium before the introduction is set to 1. Then, the adhesion time of fermented milk obtained using lactic acid bacteria into which the viscosity improving protein, the viscosity improving DNA, or the vector has been introduced is 2 or more, preferably 3 or more, and more preferably 4 or more. The adhesion time can be measured using the above creepmeter under the above conditions as the time required for the fermented milk adhering to the jig to be completely separated from the fermented milk in the container after the jig is lifted, where the moment immediately after each fermented milk is compressed twice is taken as 0 seconds.

### Lactic Acid Bacteria Composition

The lactic acid bacteria composition of the present invention is a composition containing the lactic acid bacterium of the present invention. The lactic acid bacteria composition of the present invention may further contain additional components. The additional components are not particularly limited, and examples thereof include cultures, such as the culture supernatant after completion of the culture of the lactic acid bacteria and medium components; concentrates, crudely purified products, purified products, diluted products, dried products (such as spray-dried products and freeze-dried products), frozen products, and the like of the above cultures; and protective agents, fermentation promoters, and the like, and one of these may be used alone, or a combination of two or more thereof may be used.

In addition, the lactic acid bacteria composition of the present invention includes any composition containing the lactic acid bacterium of the present invention (that is, a lactic acid bacterium into which at least one selected from the group consisting of a viscosity improving protein, a viscosity improving DNA, and a vector of the present invention containing a viscosity improving DNA has been introduced; a lactic acid bacterium having a viscosity improving DNA; a lactic acid bacterium evaluated as having a fermented milk viscosity improving effect by the method for evaluating lactic acid bacteria of the present invention (including those evaluated to have a high possibility of having the above effect) ; and a lactic acid bacterium obtained by the method for producing lactic acid bacteria of the present invention), and also includes the following fermented milk of the present invention.

### Method for Evaluating Lactic Acid Bacteria, and Method for Producing Lactic Acid Bacteria

The method for evaluating lactic acid bacteria of. the present invention is a method for evaluating whether or not lactic acid bacteria have a fermented milk viscosity improving effect, using as an indicator at least one (that is, the viscosity improving DNA of the present invention) selected from the group consisting of DNAs encoding the viscosity improving proteins of the present invention, that is, the following proteins (a) to (d):
(a) a protein which is composed of an amino acid sequence set forth in SEQ ID NO: 1;
(b) a protein which is, in the amino acid sequence set forth in SEQ ID NO: 1, composed of an amino acid sequence in which one or more amino acids other than tyrosine at position 40 are substituted, deleted, inserted, and/or added and has a fermented milk viscosity improving effect;
(c) a protein which is composed of an amino acid sequence having 80% or more identity with the amino acid sequence set forth in SEQ ID NO: 1, has tyrosine as an amino acid corresponding to position 40 of the amino acid sequence set forth in SEQ ID NO: 1, and has a fermented milk viscosity improving effect; and
(d) a protein which is composed of an amino acid sequence encoded by a DNA that hybridizes under stringent conditions with a complementary strand of a

DNA composed of the nucleotide sequence set forth in SEQ ID NO: 2, has tyrosine as an amino acid corresponding to position 40 of the amino acid sequence set forth in SEQ ID NO: 1, and has a fermented milk viscosity improving effect.

### Evaluation Step

In the method for evaluating lactic acid bacteria of the present invention, it is evaluated whether or not they have a fermented milk viscosity improving effect (evaluation step), using as an index the viscosity improving DNA of the present invention, that is, using as an index whether or not the lactic acid bacteria have the viscosity improving DNA of the present invention. When the lactic acid bacteria have the viscosity improving DNA, they can be evaluated as having a fermented milk viscosity improving effect (including evaluation as having a high possibility of having it). Meanwhile, when the lactic acid bacteria do not have the DNA, they can be evaluated as not having the fermented milk viscosity improving effect (including evaluation as having a high possibility of not having it). This makes it possible to select lactic acid bacteria that have or are likely to have a fermented milk viscosity improving effect. In the method for evaluating lactic acid bacteria of the present invention, lactic acid bacteria to be evaluated are not particularly limited, and desired lactic acid bacteria can be used as appropriate.

Whether or not lactic acid bacteria have the viscosity improving DNA of the present invention can be determined by detecting the DNA. As a method for detecting the viscosity improving DNA, a known method or a method according thereto can be appropriately employed.

For example, first, genomic DNA is extracted from the lactic acid bacterium to be evaluated. The method for extracting genomic DNA is not particularly limited, and a known method or a method according thereto can be appropriately employed. Examples include the PCI method, GuSCN/Silica method, SDS phenol method, CTAB method, and alkali treatment method. A commercially available kit can also be used as appropriate.

The method for detecting the viscosity improving DNA can be performed by then isolating a DNA corresponding to the viscosity improving DNA and determining the nucleotide sequence of the isolated DNA. Isolation of the DNA can be performed, for example, by PCR or the like using a genomic DNA as a template, using a pair of oligonucleotide primers designed to flank at least the DNA corresponding to the viscosity improving DNA. The nucleotide sequence of the isolated DNA can be determined by methods known to those skilled in the art, such as the Sanger method and the Maxam-Gilbert method. Alternatively, the nucleotide sequence of the DNA corresponding to the viscosity improving DNA may be determined directly from the genomic DNA using a next-generation sequencer or the like.

The DNA corresponding to the viscosity improving DNA preferably contains at least a site encoding an amino acid corresponding to position 40 of R1-EpsC, and a pair of oligonucleotide primers flanking this may be designed based on the nucleotide sequence of the viscosity improving DNA (such as R1-epsC) and public databases (such as Genbank). Such oligonucleotides can be designed by those skilled in the art by a known method or a method according thereto.

Another method for detecting the viscosity improving DNA is, for example, the PCR-SSP (PCR-sequence specific primer) method. In the method, the 3′-end of one of the pair of oligonucleotides constituting the primer is designed to have a base species complementary to the site encoding the specific base of the viscosity improving DNA, for example tyrosine at position 40 of R1-EpsC when the DNA to be detected is the above (a′). Amplification by PCR using a pair of oligonucleotide primers designed in this manner is limited to the case where the viscosity improving DNA of the present invention is used as a template. The site encoding tyrosine at position 40 is not amplified when genomic DNA encoding other amino acids is used as a template. Therefore, the presence or absence of such amplification can be used as an index to detect the DNA.

In addition, as another method for detecting the viscosity improving DNA, if restriction fragment length polymorphism (RFLP) can be set at position 40 of R1-EpsC or the site encoding tyrosine corresponding thereto, it can also be detected using these RFLP markers as indicators by, for example, the PCR-RFLP method (or the CAPS [Cleaved Amplified Polymorphic Sequence] method).

Another method for detecting the viscosity improving DNA is, for example, the PCR-SSCP (PCR-single-strand conformational polymorphism) method. A double-stranded DNA amplified by PCR using a pair of oligonucleotide primers designed to flank the viscosity improving DNA is denatured by treatment with heat, alkali, or the like to form a single-stranded DNA, which is thereafter subjected to polyacrylamide gel electrophoresis containing no denaturant. Then, the single-stranded DNA is folded in the gel through intramolecular interactions to form higher-order structures. Folded structure interactions vary with different base species. Therefore, the separated single-stranded DNA is detected by silver staining or radioisotope, and the gel mobility of the single-stranded DNA can be used as an index to detect the viscosity improving DNA.

Another method for detecting the viscosity improving DNA is, for example, a method using an intercalator. In this method, first, in a reaction system containing an intercalator that emits fluorescence when inserted between DNA double strands, the genomic DNA is used as a template to amplify DNA corresponding to the viscosity improving DNA. Then, the temperature of the reaction system is changed to detect fluctuations i.n the intensity of the fluorescence emitted by the intercalator, and the fluctuations in fluorescence intensity associated with the detected change in temperature as an indicator to detect the viscosity improving DNA (particularly, position 40 of R1-EpsC or a tyrosine-encoding site corresponding thereto). Such methods include the high-resolution melting curve analysis (HRM) method.

As another method for detecting the viscosity improving DNA, for example, when the DNA to be detected is (a′) above, a method using an oligonucleotide probe that hybridizes to the region containing the site encoding tyrosine at position 40 of R1-EpsC can be used. In an embodiment of this method, first, an oligonucleotide probe specifically hybridized to the site encoding tyrosine at position 40 and labeled with a reporter fluorescent dye and a quencher fluorescent dye is prepared. Next, this oligonucleotide probe is hybridized to the genomic DNA, and the DNA sample hybridized with the oligonucleotide probe is used as a template to amplify the DNA containing the site encoding tyrosine at position 40. Then, fluorescence emitted by the reporter fluorescent dye whose suppression by the quencher has been released due to degradation of the oligonucleotide probe accompanying amplification is detected. Such methods include the double-dye probe method, the so-called TaqMan (registered trademark) probe method. AS another embodiment using an oligonucleotide probe labeled with a reporter fluorescent dye and a quencher fluorescent dye, one may utilize the cycling probe method that uses the combination of a chimeric oligonucleotide (chimera of RNA and DNA) which specifically hybridizes to the viscosity improving DNA and an enzyme such as RNase H.

Another method for detecting the viscosity improving DNA includes, for example, the LAMP (Loop-Mediated Isothermal Amplification) method. In this method, a total of six regions are set, three on each side of the target site of the double-stranded DNA, and four types of primers containing these regions (two types for each side) are used to react in the presence of a strand displacement enzyme, making it possible to generate amplification origins of loop structures on both sides of the target site. Thus, subsequently, a repeating structure of mutually complementary sequences is generated on the same strand, and the target site is amplified. When the DNA to be detected is (a′) above, if the target site is the site encoding tyrosine at position 40 of R1-EpsC, the presence or absence of each modification can be detected by determining the nucleotide sequence of the amplified product. Further, when one of the six regions is the site encoding tyrosine at position 40, the target site is not amplified when there is modification, so that the DNA can be detected using the presence or absence of such amplification as an indicator.

The method for detecting the viscosity improving DNA is not limited to the above embodiment. Other known techniques can also be used in the present invention, such as denaturing gradient gel electrophoresis method (DGGE method), invader method, pyrosequencing method, single-nucleotide primer extension (SNuPE) method, allele-specific oligonucleotide (ASO) hybridization method, ribonuclease A mismatch cleavage method, DNA microarray method, and DNA array method.

Furthermore, detection of the viscosity improving DNA is preferably detection of its expression. The method for detecting the expression of the viscosity-improving DNA may be, for example, such that an mRNA or protein is extracted from the target lactic acid bacterium in a usual manner, and the mRNA or protein encoded by the viscosity improving DNA (that is, viscosity improving protein) is detected by a known technique or a method according thereto.

Methods for detecting the mRNA encoded by the viscosity improving DNA include, for example, the RT-PCR method and northern blotting method.

As a method for detecting a protein encoded by the viscosity improving DNA (viscosity improving protein), first, a protein sample is prepared from the target lactic acid bacterium, and an antibody specific to the viscosity improving protein, that is, an antibody specific to at least tyrosine at position 40 is used to perform an antigen-antibody reaction, thereby detecting the viscosity improving protein. In such a protein detection method using an antibody, for example, an antibody specific to the viscosity improving protein is added to the protein sample to perform an antigen-antibody reaction, thereby detecting the binding of the antibody to the viscosity improving protein. If an antibody specific to the viscosity improving protein is labeled, the stringiness-enhancing protein can be directly detected. If not labeled, it is further acted with a labeled molecule (such as a secondary antibody or protein A) that recognizes the antibody, and the label of the molecule can be used to indirectly detect the viscosity improving protein. Examples of such methods include immunohistochemistry (immunostaining) method, Western blotting method, ELISA method, flow cytometry, imaging cytometry, radioimmunoassay, immunoprecipitation method, and analysis method using an antibody array. The antibody may be a polyclonal antibody or a monoclonal antibody, and methods for preparing these antibodies are known to those skilled in the art.

### Kit for Use in Evaluation Method of Present Invention

As described above, the viscosity improving effect of lactic acid bacteria can be evaluated by detecting the viscosity improving DNA of the present invention. Therefore, the present invention also provides a kit including at least one drug selected from the group consisting of the following drugs (i) and (ii) for use in the above evaluation method:
(i) a drug containing an oligonucleotide having a chain length of at least 15 nucleotides that hybridizes to the viscosity improving DNA of the present invention, a transcription product thereof, or a complementary nucleotide thereof; and
(ii) a drug containing an antibody that binds to the viscosity improving protein of the present invention.

The oligonucleotide may be in the form of a primer or in the form of a probe, depending on the method for detecting the viscosity improving DNA.

The primer is not particularly limited as long as it hybridizes to the viscosity improving DNA of the present invention or a DNA corresponding to the viscosity improving DNA, a complementary nucleotide thereof (including cDNA and cRNA), or the transcription product (mRNA) of the viscosity improving DNA, and enables amplification and detection thereof. The primer may be a DNA alone, or may be partially or wholly substituted with an artificial nucleic acid (modified nucleic acid) such as a crosslinked nucleic acid. The size of the primer is at least about 15 nucleotides long, preferably 15 to 100 nucleotides long, more preferably 18 to 50 nucleotides long, and further preferably 20 to 40 nucleotides long. Such primers can be designed and produced by methods known to those skilled in the art, according to the detection method described above.

The probe is not particularly limited as long as it hybridizes to the viscosity improving DNA or a DNA corresponding to the viscosity improving DNA, a complementary nucleotide thereof, or the transcription product of the viscosity improving DNA, and enables detection thereof. The probe may be a DNA, an RNA, an artificial nucleic acid, a chimeric molecule thereof, or the like. The probe may be single-stranded or double-stranded. The size of the probe is at least about 15 nucleotides long, preferably 15 to 1000 nucleotides long, more preferably 20 to 500 nucleotides long, and further preferably 30 to 300 nucleotides long. Such probes can be designed and produced by methods known to those skilled in the art. Also, the probe may be provided in a form immobilized on a substrate, such as a microarray.

The antibody is not particularly limited as long as it can specifically bind to the viscosity improving protein of the present invention. For example, it may be either a polyclonal antibody or a monoclonal antibody, and may be a functional fragment of an antibody (such as Fab, Fab′, or scFv). Such antibodies can be produced by methods known to those skilled in the art. In addition, the antibody may be provided in a form immobilized on a substrate, such as a plate for use in ELISA, antibody arrays, and the like.

Moreover, the oligonucleotide or antibody contained in the kit may be labeled with a labeling substance in accordance with the detection method described above. Examples of the labeling substance include fluorescent substances such as FITC, FAM, DEAC, R6G, TexRed, and Cy5, enzymes such as β-D-glucosidase, luciferase, HRP, radioactive isotopes such as ³H, ¹⁴C, ³²P, ³⁵S, ¹²³I, affinity substances such as biotin and streptavidin, and luminescent substances such as luminol, luciferin, and lucigenin.

The method for evaluating lactic acid bacteria of the present invention may further include 10 confirmation step of confirming whether or not the lactic acid bacteria have a fermented milk viscosity improving effect. Such confirmation method is not particularly limited, but the fermented milk obtained using the lactic acid bacteria to be evaluated can be confirmed, for example, by using the adhesion time measured using the creepmeter under the above conditions as an index. Examples of the fermented milk include those obtained by seeding 1% (wt/wt) of the lactic acid bacteria in a 10% powdered skim milk medium and fermenting the culture overnight at 37° C. under anaerobic conditions. In addition, the ones with the adhesion time of, for example, 2 seconds or longer, preferably 3 seconds or longer, and further preferably 4 seconds or longer can be evaluated that the lactic acid bacteria have a fermented milk viscosity improving effect.

### Method for Producing Lactic Acid Bacteria

The method for producing lactic acid bacteria of the present invention is a method including :
an evaluation step of evaluating whether or not the lactic acid bacteria have a fermented milk viscosity improving effect by the method for evaluating lactic acid bacteria of the present invention; and
a step of obtaining lactic acid bacteria evaluated as having a fermented milk viscosity improving effect in the evaluation step.

In the method for producing lactic acid bacteria of the present invention, examples of the evaluation step include the Evaluation Step of <Method for Evaluating Lactic Acid Bacteria> described above. The evaluation step according to the method for producing lactic acid bacteria of the present invention selects lactic acid bacteria that have or are likely to have a fermented milk viscosity improving effect. In the method for producing lactic acid bacteria of the present invention, the evaluation step makes it possible to obtain lactic acid bacteria evaluated as having a fermented milk viscosity improving effect (including those evaluated to have a high possibility of having the above effect), and for example, by culturing the selected lactic acid bacteria in an appropriate medium, those lactic acid bacteria may be obtained as a culture thereof.

Moreover, the form of the lactic acid bacteria obtained by the method for producing lactic acid bacteria of the present invention may be the form of a lactic acid bacteria composition such as a culture thereof. Therefore, the method for producing lactic acid bacteria of the present invention also includes a method for producing a lactic acid bacteria composition, which includes a step of obtaining a lactic acid bacteria composition containing the lactic acid bacteria evaluated as having a fermented milk viscosity improving effect in the evaluation step. Additional components that may be contained in the lactic acid bacteria composition other than the lactic acid bacteria are as described above.

### Method for Producing Fermented Milk

The method for producing fermented milk of the present invention includes a fermentation step of adding lactic acid bacteria or a lactic acid bacteria composition to a milk preparation solution containing raw material milk and fermenting the mixture.

The lactic acid bacteria according to the method for producing fermented milk of the present invention include the above-described lactic acid bacteria of the present invention (that is, a lactic acid bacterium into which at least one selected from the group consisting of a viscosity improving protein, a viscosity improving DNA, and a vector of the present invention containing a viscosity improving DNA has been introduced; a lactic acid bacterium having a viscosity improving DNA; a lactic acid bacterium evaluated as having a fermented milk viscosity improving effect by the method for evaluating lactic acid bacteria of the present invention (including those evaluated to have a high possibility of having the above effect); and a lactic acid bacterium obtained by the method for producing lactic acid bacteria of the present invention), and one of these may be used alone, or two or more may be used in combination. In addition, the lactic acid bacteria composition according to the method for producing fermented milk of the present invention includes the above-described lactic acid bacteria composition of the present invention; and the lactic acid bacteria composition obtained by the method for producing lactic acid bacteria of the present invention, and one of these may be used alone, or two or more may be used in combination. By using these lactic acid bacteria lactic acid bacteria compositions, fermented milk excellent in viscosity can be obtained. Note that in the case of using lactic acid bacteria evaluated as having a fermented milk viscosity improving effect by the method for evaluating lactic acid bacteria of the present invention, the method for producing fermented milk of the present invention may include the above evaluation step, but in this case, the evaluation step may be performed only once at the beginning.

In the method for producing fermented milk of the present invention, additional lactic acid bacteria other than the lactic acid bacterium of the present invention may be further used in combination. Moreover, yeast may be further added. Examples of the additional lactic acid bacteria and yeast include lactic acid bacteria and yeast that are conventionally known to be contained in fermented milk.

### (Milk Preparation Solution)

The milk preparation solution according to the present invention contains raw material milk. The raw material milk preferably contains lactose, and examples thereof include raw milk (such as milk of cow, water buffalo, sheep, goat, and the like), pasteurized milk, whole milk, skim milk, whey, and processed products thereof (such as whole milk powder, full-fat concentrated milk, skimmed milk powder, skimmed concentrated milk, condensed milk, whey powder, buttermilk, butter, cream, cheese, whey protein concentrate (WPC), whey protein isolate (WPI), α-lactalbumin (α-La), and β-lactoglobulin (β-Lg)), and may be one of these or a mixture of two or more thereof.

The milk preparation solution according to the present invention may be composed only of the above raw material milk, or may be an aqueous solution, diluted solution, or concentrated solution of the above raw material milk, or may further contain additional components in addition to the above raw material milk, if necessary. Examples of the additional components include water; foods, food ingredients, and food additives such as soymilk, saccharides such as sugar, sweeteners, flavors, fruit juices, fruit pulps, vitamins, minerals, oils and/or fats, ceramides, collagen, milk phospholipids, yeast extracts, and polyphenols ; stabilizers, thickeners, and gelling agents such as pectin, soy polysaccharides, CMC (carboxymethylcellulose), agar, gelatin, carrageenan, and gums, and may be one of these or a mixture of two or more thereof. The milk preparation solution can be prepared by mixing the above components, while optionally with heating and/or optionally with homogenizing. In addition, as the milk preparation solution, heat-sterilized one can also be used.

### (Fermentation)

As the fermentation step of adding the lactic acid bacteria or lactic acid bacteria composition to the milk preparation solution and fermenting the mixture, a known method or a method according thereto can be appropriately employed, and is not particularly limited. Examples thereof include a method of seeding the lactic acid bacteria or lactic acid bacteria composition as a fermentation starter into the milk preparation solution and fermenting the mixture. The lactic acid bacteria or lactic acid bacteria composition is preferably added to the milk preparation solution in the form of the lactic acid bacteria composition, more preferably in the form of a culture or culture concentrate.

The amount of the fermentation starter to be added can be appropriately set according to the amount of addition employed in known methods for producing fermented milk, and for example, it is preferably 1×10⁷ to 5×10⁹ CFU/mL, more preferably 1×10³ to 2×10⁹ CFU/mL, based on the volume of the milk preparation solution in terms of lactic acid bacteria count (total bacteria count in the case of a combination of two or more). Also, it is preferably 0.1 to 2% (wt/wt), more preferably 0.5 to 1.5% (wt/wt), and further preferably 0.5 to 1% (wt/wt), based on the volume of the milk preparation solution.

The method of seeding the fermentation starter is not particularly limited, and a method commonly used in the production of fermented milk can be appropriately used. The fermentation conditions are not particularly limited, and can be appropriately selected depending on the growth conditions of lactic acid bacteria to be added, the amount of the milk preparation solution, and the like. For example, under aerobic or anaerobic conditions at a temperature of 35 to 45° C. and more preferably at a temperature of 38 to 43 ° C., the mixture is allowed to stand or stirred (preferably stand) for usually 3 to 24 hours, more preferably 3 to 8 hours, and further preferably 4 to 6 hours until the pH of the milk preparation solution added with the above lactic acid bacteria or lactic acid bacteria composition is 4.8 or less, more preferably 4.0 to 4.6. In addition, as the anaerobic conditions, for example, fermentation under nitrogen aeration conditions can be employed.

The fermented milk of the present invention can be obtained by the above fermentation. The fermented product after the fermentation step (that is, the milk preparation solution and the lactic acid bacteria or lactic acid bacteria composition after the fermentation step) can be used as the fermented milk of the present invention as it is or by concentrating, diluting, drying, freezing, or the like as necessary. Also, the fermented milk of the present invention may be obtained by crushing or heat-treating the lactic acid bacteria in the fermented product, or by concentrating, diluting, drying, freezing, or the like as necessary.

### Fermented Milk

As the fermented milk of the present invention, fermented milk containing at least one lactic acid bacterium selected from the group consisting of the above-described lactic acid bacteria of the present invention (that is, a lactic acid bacterium into which at least one selected from the group consisting of a viscosity improving protein, a viscosity improving DNA, and a vector of the present invention containing a viscosity improving DNA has been introduced; a lactic acid bacterium having a viscosity improving DNA; a lactic acid bacterium evaluated as having a fermented milk viscosity improving effect by the method for evaluating lactic acid bacteria of the present invention (including those evaluated to have a high possibility of having the above effect); and a lactic acid bacterium obtained by the method for producing lactic acid bacteria of the present invention) is provided. The fermented milk of the present invention preferably contains a viscosity improving protein and/or exopolysaccharide derived from these lactic acid bacteria. In addition, the fermented milk of the present invention may further contain additional lactic acid bacteria and yeast.

The fermented milk of the present invention is not particularly limited, and examples may be any of fermented milk that satisfies the standards for “fermented milk” according to the Ministerial Ordinance on Milk and Milk Products Concerning Compositional Standards, etc. (Ministerial Ordinance on Milk and Milk Products) issued by the Ministry of Health, Labor and Welfare of Japan (more specifically, the content of milk solids-not-fat is 8.0% or more, and the lactic acid bacteria count or yeast count (preferably the lactic acid bacteria count) is 10 million/mL or more), those that satisfy the standards for “milk products and fermented milk drinks” (more specifically, the content of milk solids-not-fat is 3.0% or more, and the lactic acid bacteria count or yeast count (preferably the lactic acid bacteria count) is 10 million/mb or more), those that satisfy the standards for “fermented milk drinks” (more specifically, the content of milk solids-not-fat is 3.0% or more, and the lactic acid bacteria count or yeast count (preferably the lactic acid bacteria count) is 1 million/mL or more). Note that the milk solids-not-fat refers to the remaining components (mainly such as proteins, lactose, and minerals) obtained by subtracting the fat content from the total milk solids, and the lactic acid bacteria and yeast count are measured by the test method specified in the above Ministerial Ordinance on Milk and Milk Products before the pasteurization.

The fermented milk of the present invention may be a fermented product after the fermentation step or may be obtained by pasteurizing the fermented product, or may be obtained by, for example, concentrating, diluting, drying, or freezing them. For example, the fermented milk may be a pasteurized product of the above-mentioned fermented milk, milk products and fermented milk drinks, or fermented milk drinks, and in this case, the lactic acid bacteria count is in terms of viable bacteria count. The lactic acid bacteria contained in the fermented milk of the present invention include not only viable bacteria but also dead bacteria, as well as crushed products and heat-treated products of lactic acid bacteria, and concentrates, crudely purified products, purified products, dilutions, dried products (such as spray-dried products and freeze-dried products), and frozen products thereof. The lactic acid bacteria contained in the fermented milk of the present invention preferably include at least viable bacteria.

The fermented milk of the present invention may further contain the above-described additional lactic acid bacteria or yeast as lactic acid bacteria as long as the effects of the present invention are not impaired. In addition, the fermented milk of the present invention may further contain various components that can be contained in food and drink. Such components are not particularly limited, and examples thereof include water, saccharides, sugar alcohols, minerals, vitamins, proteins, peptides, amino acids, organic acids, pH adjusters, starches and modified starches, dietary fibers, fruits and vegetables and processed products thereof, animal and plant crude drug extracts, naturally-derived polymers (such as collagen, hyaluronic acid, and chondroitin), oils and/or fats, thickeners, emulsifiers, solvents, surfactants, gelling agents, stabilizers, buffers, suspending agents, thickening agents, excipients, disintegrators, binders, flow agents, preservatives, colorants, flavors, corrigents, and sweeteners. One of these may be contained alone, or two or more thereof may be contained in combination.

Such fermented milk is preferably yogurt, cheese, fermented cream, fermented butter, or the like, and particularly preferably yogurt. Specific examples of the yogurt include set type yogurt (solid fermented milk) such as plain yogurt, soft-type yogurt (pasty fermented milk), and drink type yogurt (liquid fermented milk), and frozen yogurt using these ingredients may also be used. In addition, the fermented milk of the present invention can also be used as an ingredient for fermented food such as cheese, fermented cream, fermented butter, and kefir.

The fermented milk of the present invention can be obtained by the method for producing fermented milk of the present invention, and can be fermented milk excellent in viscosity.

### Method for Producing Exopolysaccharide

The present invention also provides a method for producing an exopolysaccharide of lactic acid bacteria, including a step of adding the lactic acid bacterium or lactic acid bacteria composition of the present invention to a medium containing glucose and/or a saccharide composed of glucose, fermenting the mixture, and collecting an exopolysaccharide contained in a fermented product.

In the present invention, “exopolysaccharide of lactic acid bacteria” refers to an exopolysaccharide produced by lactic acid bacteria, and such exopolysaccharides include neutral exopolysaccharides (NPS), acidic exopolysaccharides (APS), zwitterionic exopolysaccharides (ZPS), and mixtures thereof.

The medium must contain at least one saccharide selected from glucose and saccharides composed of glucose. Examples of saccharides composed of glucose include disaccharides (such as maltose, sucrose, and lactose), oligosaccharides (such as galacto-oligosaccharides, fructo-oligosaccharides, and mannan-oligosaccharides), and polysaccharides (such as starch (amylose, amylopectin) and glycogen). The saccharide contained in the medium may be one of the above saccharides or a combination of two or more of them, and among them, lactose is preferably contained. In addition, as the saccharide contained in the medium, for example, saccharide contained in the raw material milk can be used. The medium preferably contains the raw material milk, and more preferably is the milk preparation solution containing the raw material milk. Moreover, as the raw material milk, skim milk powder is preferable.

The lactic acid bacteria and lactic acid bacteria composition as well as the fermentation method are the same as in the fermentation step in the method for producing fermented milk described above, including the preferable aspects thereof, except that the medium may be used as the milk preparation solution. A method for collecting the exopolysaccharide from the fermented milk is not particularly limited, and a conventionally known method or a method according thereto can be appropriately employed. Examples thereof include a method in which a fermented product after fermentation is, if necessary, subjected to deproteinization by addition of a protein denaturant (such as trichloroacetic acid) or heat treatment to obtain a crudely purified product, which is purified by, for example, salting-out method, organic solvent precipitation method, membrane separation method, or chromatographic separation method, either alone or in combination of two or more thereof.

### Fermented Milk Thickener and Method for Producing the Same

The present invention also provides a fermented milk thickener including, as an active ingredient, an exopolysaccharide derived from at least one lactic acid bacterium selected from the group consisting of the above-described lactic acid bacteria of the present invention (that is, a lactic acid bacterium into which at least one selected from the group consisting of a viscosity improving protein, a viscosity improving DNA, and a vector of the present invention containing a viscosity improving DNA has been introduced; a lactic acid bacterium having a viscosity improving DNA; a lactic acid bacterium evaluated as having a fermented milk viscosity improving effect by the method for evaluating lactic acid bacteria of the present invention (including those evaluated to have a high possibility of having the above effect) ; and a lactic acid bacterium obtained by the method for producing lactic acid bacteria of the present invention). The exopolysaccharide derived from lactic acid bacteria is produced extracellularly when the lactic acid bacterium or lactic acid bacteria composition of the present invention is added to the medium and fermented by the method for producing an exopolysaccharide, and is contained in the fermented product after fermentation.

The fermented milk thickener of the present invention can, if added to fermented milk for example, improve the viscosity of the fermented milk to thicken it, for example.

The fermented milk thickener of the present invention may be the fermented product after fermentation, or may be a concentrate, crudely purified product, purified product, paste product, dried product (such as a spray-dried product or freeze-dried product), crushed product, or medium-dispersed liquid of the above-described fermented product, or may be a treated product that combines two or more of these, or may be composed only of the exopolysaccharide obtained by the method for producing an exopolysaccharide. Furthermore, it may contain additional components that can be contained in fermented milk, as long as the effects of the present invention are not impaired. The additional components are not particularly limited, and examples thereof include the various components listed in <Fermented Milk> described above, and one or more of these may be contained in an appropriate amount in combination.

In the fermented milk thickener of the present invention, although it cannot be said unconditionally because it is adjusted as appropriate, the content of the exopolysaccharide, which is an active ingredient (total amount thereof in the case of a mixture of two or more) is preferably 0.001% by mass or more, more preferably 0.002% by mass or more, and particularly preferably 0.003% by mass or more, based on the total fermented milk thickener. The upper limit of the content is not particularly limited, and can be, for example, 100% by mass or less, preferably 90% by mass or less.

### Method for Improving Viscosity of Fermented Milk

The method for improving the viscosity of fermented milk of the present invention is a method including a fermentation step of adding the lactic acid bacterium or lactic acid bacteria composition of the present invention to the milk preparation solution containing raw material milk and fermenting the mixture. This makes it possible to improve the viscosity of fermented milk. The lactic acid bacteria, lactic acid bacteria composition, and fermentation step described above are as stated in the aforementioned method for producing fermented milk of the present invention. This makes it possible to improve the viscosity of fermented milk.

In the present invention, whether fermented milk has excellent or improved viscosity can be evaluated, for example, by comparing the adhesion time of fermented milk with the adhesion time of fermented milk obtained under the same fermentation conditions using a lactic acid bacterium other than the lactic acid bacteria described above, that is, an additional lactic acid bacterium not having any of the DNAs (a′) to (d′), preferably at least one lactic acid bacterium selected from the group consisting of Lactobacillus delbrueckii and Lactobacillus delbrueckii subspecies bulgaricus. For example, when the adhesion time of fermented milk obtained using the above additional lactic acid bacteria is set to 1, if the adhesion time of the target fermented milk is 2 or more, preferably 3 or more, or more preferably 4 or more, it can be evaluated that the fermented milk has excellent or improved viscosity. Alternatively, if the adhesion time of the target fermented milk is, for example, 2 seconds or more, preferably 3 seconds or more, or more preferably 4 seconds or more, it can be evaluated that the fermented milk has excellent or improved viscosity.

The adhesion time can be measured using the above creepmeter under the above conditions as the time required for the fermented milk adhering to the jig to be completely separated from the fermented milk in the container after the jig is lifted, where the moment immediately after each fermented milk is compressed twice is taken as 0 seconds. In addition, whether the above additional lactic acid bacteria do not have any of the above DNAs (a′) to (d′) can be confirmed by the method for detecting viscosity improving DNA described in the Evaluation Step of <Method for Evaluating Lactic Acid Bacteria>described above. As a lactic acid bacterium not having any of the above DNAs (a′) to (d′), specifically, for example, Lactobacillus delbrueckii subspecies bulgaricus 2038 strain (2038 strain) is preferable.

### EXAMPLES

The present invention will be described in more detail based on Examples below, but the present invention is not limited to the following Examples.

### Lactic Acid Bacteria

The lactic acid bacteria used in the following tests are as follows.

R-1 strain: Lactobacillus delbrueckii subsp. bulgaricus OLL1073R-1 (accession number: FERM BP-10741) 2038 strain: Lactobacillus delbrueckii subsp. bulgaricus 2038

Note that the 2038 strain is a strain that is isolated by smearing a diluted solution of Meiji Bulgaria Yogurt LB81 (manufactured by Meiji Co., Ltd.) on a BCP agar medium, culturing at 37ºC for 48 hours, and then picking up rough colonies, and the full -length genome of the 2038 strain is registered in Kyoto

Encyclopedia of Genes and Genomes (KEGG), an integrated database of biological system information that integrates information on genomes, proteins, and compounds through intermolecular interactions, reactions, and relational networks, under entry number : T01957.

### Viscosity Evaluation 1

### (1) Preparation of Fermented Milk

To a 10% powdered skim milk medium prepared with 10% (wt/wt) non-fat dry milk, 0.1% (wt/wt) yeast extract, and distilled water, the R-1 strain was seeded to 1% (wt/wt) and fermented overnight at 37° C. under anaerobic conditions to obtain fermented milk. Fermented milk was also obtained under the same conditions except that the 2038 strain was used instead of the R-1 strain.

### (2) Adhesion Time Measurement

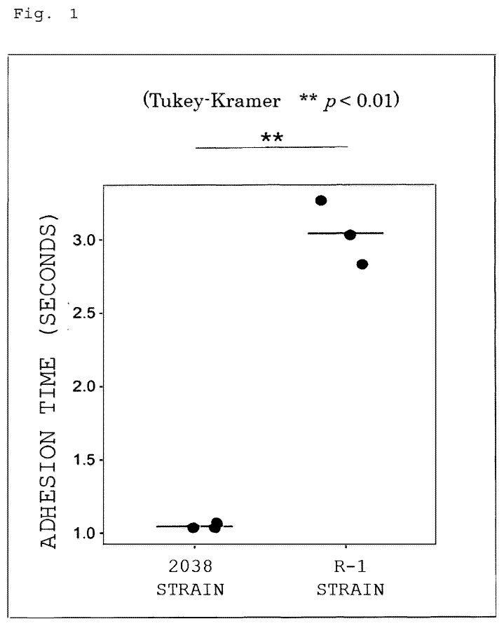

Each of the fermented milks obtained in (1) above was subjected to viscosity evaluation using a creepmeter (model: RE2-33005S (manufactured by Yamaden Co., Ltd.), container: a cylindrical container with φ =41 mm and height=35 mm, jig: φ=25.2 mm, height=25 mm, rate: 10 mm/sec, return distance: 10 mm, amount of fermented milk: 10 g). Specifically, the adhesion time (seconds) was obtained by measuring the time required for the fermented milk adhering to the jig to be completely separated from the fermented milk in the container after the jig was lifted, where the moment immediately after the obtained fermented was compressed twice using the above creepmeter was taken as 0 seconds. The measurement was performed 3 times for each fermented milk, and the average value was calculated. It can be evaluated that the longer the adhesion time is, the higher and better the viscosity is. FIG. 1 shows the results. As shown in FIG. 1, compared to the average adhesion time of the fermented milk fermented with the 2038 strain, the average adhesion time of the fermented milk fermented with the R-1 strain was significantly longer (p<0.01 in Tukey-Kramer test), indicating high viscosity.

### Comparison of EPS Gene Cluster Regions

The nucleotide sequence of the 2038 strain genome registered in KEGG was used to extract the region −100 bp to +100 bp for each of the two EPS gene cluster regions: LBU1598-LBU1588 (EPS gene cluster 1) and LBU1630-LBU1618 (EPS gene cluster 2). In addition, the full-length genome of the R-1 strain was obtained using the next-generation sequencer Miseq (manufactured by Illumina). Homology/Local BLASTN from Genetyx Ver.13 (manufactured by GENETYX CORPORATION) was used to extract nucleotide sequences homologous to the above two EPS gene cluster regions from the genome of the R-1 strain (E-value threshold=0.00001, word size=11).

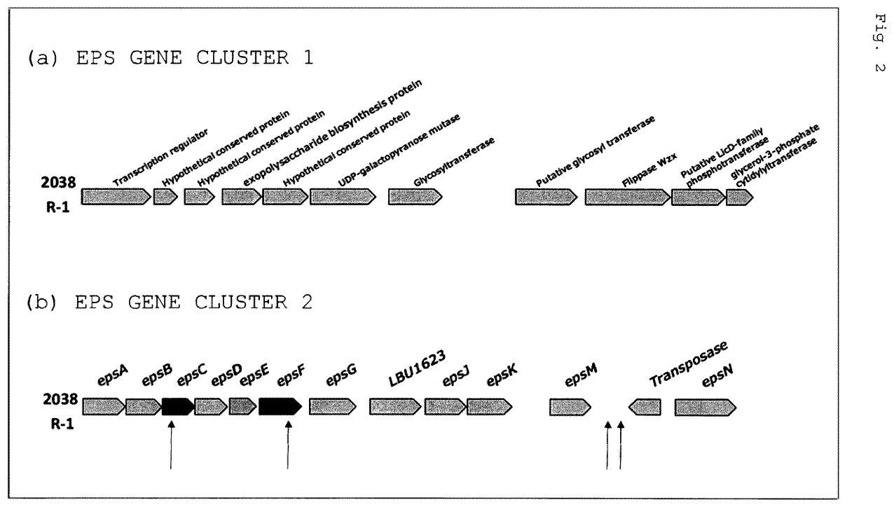

FIG. 2 (a) shows a schematic diagram of EPS gene cluster 1, and FIG. 2 (b) shows a schematic diagram of EPS gene cluster 2. In FIG. 2, each arrow indicates a location where a difference in the nucleotide sequences was found between the 2038 strain and the R-1 strain. EPS gene clusters 1 and 2 were also highly conserved in the R-1 strain genome. In particular, EPS gene cluster 1 completely matched the nucleotide sequence of the 2038 strain over the entire region (11,569 bp). Meanwhile, as shown in FIG. 2, in EPS gene cluster 2, among 15,769 bp, a total of 4 base differences were observed in the epsC gene, the epsF gene, and the intergenic regions between the between the epsM gene and Transposase gene. Table 1 shows the genes or regions found to be different between the 2038 strain genome and the R-1 strain genome, the bases, the codons containing the bases, the amino acids encoded by the codons, and the positions of the amino acids in the genes. In addition, since there was a frameshift due to base differences in the epsF gene, Table 2 below shows the nucleotide sequences after the codons containing the bases found to be different between the 2038 strain genome and the R-1 strain genome, and the amino acid sequences encoded by them. In addition, the nucleotide sequence of the 2038 strain genome presented in Table 2 is set forth in SEQ ID NO: 3, and the amino acid sequence is set forth in SEQ ID NO: 4; and the nucleotide sequence of the R-1 strain genome is set forth in SEQ ID NO: 5, and the amino acid sequence is set forth in SEQ ID NO: 6.

TABLE 1

| | 2038 Strain | | | | R-1 Strain | | |
|---|---|---|---|---|---|---|---|
| Gene/Region | Base | Codon | Amino Acid | Amino Acid Position | Base | Codon | Amino Acid |
| epsC | A | AAU | Asparagine | Position 40 | T | UAU | Tyrosine |
| epsF | G | GGG | Glycine | Position 333 | del | GGC | Glycine |
| Intergenic 1 | A | — | — | — | T | — | — |
| Intergenic 2 | G | — | — | — | A | — | — |

TABLE 2

| Strain | Nucleotide Sequence (5′-3′) | Amino Acid Sequence (N-C) |
|---|---|---|
| 2038 | GGGCTCGCTATTCTCTGA | GLAIL |
| R-1 | GG-CTCGCTATTCTCTGATTGA | GSLFSD |

As presented in Table 1, the first difference was that adenine (A) at base 118 of the spsC gene in the 2038 strain genome was replaced with thymine (T) in the R-1 strain genome. In addition, the amino acid at position 40 specified by the codon containing this base was asparagine in the 2038 strain genome, but was tyrosine in the R-1 strain genome. The second difference was that the guanine (G) at base 999 of the epsF gene in the 2038 strain genome was deleted in the R-1 strain genome. As a result, in the R-1 strain genome, there was a frame shift, and the amino acid specified by the codon containing this base was glycine (G) at position 333 in both the 2038 strain genome and the R-1 strain genome. However, as presented in Table 2, the frameshift resulted in different amino acid sequences from the glycine to the C terminus between the two strains, where the 2038 strain genome had N terminus-Gly-Leu-Ala-Ile-Leu-C terminus and the R-1 strain genome had N terminus-Gly-Ser-Leu-Phe-Ser-Asp-C terminus. The third and fourth differences were the intergenic regions (intergenic 1, 2) between the epsM gene and Transposase gene. Since mutations in intergenic regions did not appear to involve any gene, the base difference in either the epsC gene or epsF gene and the accompanying difference in amino acid composition were presumed to be involved in the difference in viscosity between the fermented milk fermented with the R-1 strain and the fermented milk fermented with the 2038 strain.

### Viscosity Evaluation 2

(1) Preparation of the 2038-epsC Strain and 2038-epsF Strain by Transformation of the 2038 Strain

A shuttle vector pGMβ1 (obtained from Meiji University) for Escherichia coli and lactic acid bacteria was used for transformation of the 2038 strain. pGMβ1 was introduced into Escherichia coli DHS (E. coli DHS) by electroporation method. LB medium containing ampicillin (final concentration of 50 μg/mL) and erythromycin (final concentration of 500 μg/ml) was used to select E. coli DH5 carrying pGMβ1, and then plasmid extraction was performed to purify pGMβ1. Also, PCR was used to amplify the epsC gene and epsF gene of the R-1 strain using the following primers:
epsC gene:
   Lb_epsC_rec1_F (nucleotide sequence set forth in SEQ ID NO: 7)
   Lb_epsC_rec1_R (nucleotide sequence set forth in SEQ ID NO: 8)
epsF gene:
   Lb epsF rec1 F (nucleotide sequence set forth in SEQ ID NO: 9)
   Lb_epsF_rec1_R (nucleotide sequence set forth in SEQ ID NO: 10).

The purified pGMß1 was then subjected to SacI or SalI treatment for dephosphorylation. Further, the amplified product of the epsC gene of the R-1 strain was treated with SacI, and the amplified product of the epsF gene was treated with SalI, and each of these was ligated with the aforementioned pGMβ1 to obtain plasmids “pGMβ1-epsC” and “pGMβ1-epsF”. The electroporation method was used to introduce pGMβ1-epsC or pGMβ1-epsF into E. coli DH5. LB medium containing ampicillin (final concentration of 50 μg/mL) and erythromycin (final concentration of 500 μg/mL) was used to select E. coli DHS carrying pGMβ1-epsC or pGM61-epsF, and then plasmid extraction was performed to purify pGMβ1-epsC and pGMβ1-epsF. Next, the electroporation method was used to introduce pGMβ1-epsC or pGMβ1-epsF into Lactococcus lactis IL1403 (L. lactis IL1403). Then, on a 0.45 μm membrane filter placed in a Buchner flask, the culture medium of L. lactis IL1403 carrying pGMβ1-epsC or pGMβ1-epsF and the culture medium of 2038 strain were aspirated, and pGMβ1-epsC or pGMβ1 -epsF was introduced into the 2038 strain by conjugative transfer. By culturing at 40° C.using MRS medium containing erythromycin (final concentration of 25 μg/mb), the 2038 strain carrying pGMβ1-epsC or pGMβ1-epsF was selected. Subsequently, subculturing in MRS medium was repeated to obtain the 2038 strain introduced with the epsC gene of the R-1 strain (hereinafter Example 1: “2038-epsC strain”) and the 2038 strain introduced with the epsF gene of the R-1 strain (hereinafter Comparative Example 1: “2038-epsF strain”).

### (2) Preparation of Fermented Milk

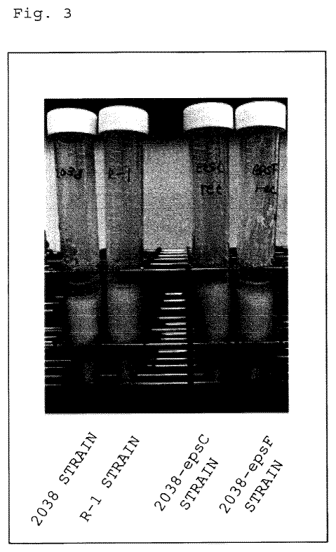

To a 10% powdered skim milk medium prepared with 10% (wt/wt) non-fat dry milk, 0.1% (wt/wt) yeast extract, and distilled water, the 2038-epsC strain and the 2038-epsF strain were each seeded to 1% (wt/wt) and fermented overnight at 37° C. under anaerobic conditions to obtain fermented milk. Further, each fermented milk was obtained in the same manner for the R-1 strain and the 2038 strain. FIG. 3 shows the appearance of fermented milk obtained using each strain. As shown in FIG. 3, compared to the fermented milk fermented with the 2038 strain and the 2038-epsF strain, the fermented milk fermented with the R-1 strain or the 2038-epsC strain showed almost no separation of whey.

### (3) Adhesion Time Measurement

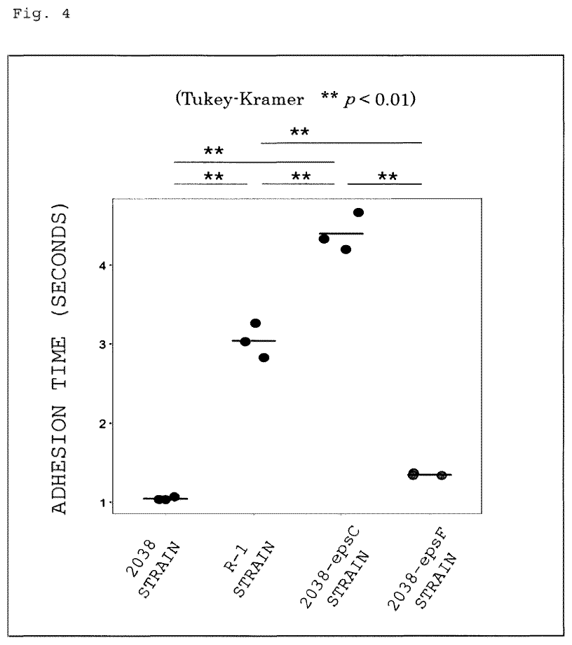

For each fermented milk obtained in (2) of <Viscosity Evaluation 2> above, the adhesion time was measured in the same manner as the adhesion time measurement in (2) of <Viscosity Evaluation 1>, and the viscosity was evaluated. The adhesion time measurement was performed 3 times for each fermented milk, and the average value was calculated. FIG. 4 shows the results. As shown in FIG. 4, compared to the average adhesion time of the fermented milk fermented with the 2038 strain and 2038-epsF strain, the average adhesion time of the fermented milk fermented with the 2038-epsC strain was significantly longer (p <0.01 in Tukey-Kramer test), indicating high viscosity. As described above, it was confirmed that introducing the DNA of the epsc gene of the R-1 strain into lactic acid bacteria could improve the viscosity of the resulting fermented milk. In addition, a difference was observed only in the amino acid at position 40 between the epsC gene of the R-1 strain, which exhibited high viscosity, and the epsC gene of the 2038 strain, which exhibited inferior viscosity. It can thus be said that the tyrosine at position 40 of the protein encoded by the epsC gene is important for the fermented milk viscosity improving effect.

### INDUSTRIAL APPLICABILITY

As described above, the present invention makes it possible to provide a novel protein having a fermented milk viscosity improving effect, fermented milk excellent in viscosity, and a method for producing the same. More specifically, it becomes possible to all provide a novel protein having a fermented milk viscosity improving effect, a DNA encoding the protein, a vector containing the DNA, a lactic acid bacterium containing the DNA or the vector, and a lactic acid bacteria composition thereof, as well as fermented milk excellent in viscosity and a fermented milk thickener, and a method for producing them, a method for improving the viscosity of fermented milk, and a method for evaluating lactic acid bacteria.

For example, by introducing a DNA encoding the novel protein of the present invention into various lactic acid bacteria, it becomes possible to easily produce fermented milk with higher viscosity and thickness than conventional ones. In addition, by using the sequence of a DNA encoding the novel protein of the present invention as a selection criterion, it becomes possible to easily select lactic acid bacteria capable of producing fermented milk with higher viscosity and thickness than conventional ones. Furthermore, the novel protein of the present invention can improve the viscosity of fermented milk, thereby reducing the amount of free whey and improving the appearance of the fermented milk.

1. A composition comprising: at least one protein selected from the group consisting of the following proteins (a) to (d):
(a) a protein which is composed of an amino acid sequence set forth in SEQ ID NO: 1;
(b) a protein which is, in the amino acid sequence set forth in SEQ ID NO: 1, composed of an amino acid sequence in which one or more amino acids other than tyrosine at position 40 are substituted, deleted, inserted, and/or added and has a fermented milk viscosity improving effect;
(c) a protein which is composed of an amino acid sequence having 80% or more identity with the amino acid sequence set forth in SEQ ID NO: 1, has tyrosine as an amino acid corresponding to position 40 of the amino acid sequence set forth in SEQ ID NO: 1, and has a fermented milk viscosity improving effect; and
(d) a protein which is composed of an amino acid sequence encoded by a DNA that hybridizes under stringent conditions with a complementary strand of a DNA composed of the nucleotide sequence set forth in SEQ ID NO: 2, has tyrosine as an amino acid corresponding to position 40 of the amino acid sequence set forth in SEQ ID NO: 1, and has a fermented milk viscosity improving effect.

2. A composition comprising: at least one selected from the group consisting of DNAs encoding the proteins (a) to (d):
(a) a protein which is composed of an amino acid sequence set forth in SEQ ID NO: 1;
(b) a protein which is, in the amino acid sequence set forth in SEQ ID NO: 1, composed of an amino acid sequence in which one or more amino acids other than tyrosine at position 40 are substituted, deleted, inserted, and/or added and has a fermented milk viscosity improving effect;
(c) a protein which is composed of an amino acid sequence having 80% or more identity with the amino acid sequence set forth in SEQ ID NO: 1, has tyrosine as an amino acid corresponding to position 40 of the amino acid sequence set forth in SEQ ID NO: 1, and has a fermented milk viscosity improving effect; and
(d) a protein which is composed of an amino acid sequence encoded by a DNA that hybridizes under stringent conditions with a complementary strand of a DNA composed of the nucleotide sequence set forth in SEQ ID NO: 2, has tyrosine as an amino acid corresponding to position 40 of the amino acid sequence set forth in SEQ ID NO: 1, and has a fermented milk viscosity improving effect.

3. A vector comprising: at least one selected from the group consisting of DNAs encoding the proteins (a) to (d):
(a) a protein which is composed of an amino acid sequence set forth in SEQ ID NO: 1,
(b) a protein which is, in the amino acid sequence set forth in SEQ ID NO: 1, composed of an amino acid sequence in which one or more amino acids other than tyrosine at position 40 are substituted, deleted, inserted, and/or added and has a fermented milk viscosity improving effect
(c) a protein which is composed of an amino acid sequence having 80% or more identity with the amino acid sequence set forth in SEQ ID NO: 1, has tyrosine as an amino acid corresponding to position 40 of the amino acid sequence set forth in SEQ ID NO: 1, and has a fermented milk viscosity improving effect and
(d) a protein which is composed of an amino acid sequence encoded by a DNA that hybridizes under stringent conditions with a complementary strand of a DNA composed of the nucleotide sequence set forth in SEQ ID NO: 2, has tyrosine as an amino acid corresponding to position 40 of the amino acid sequence set forth in SEQ ID NO: 1, and has a fermented milk viscosity improving effect.

4. (canceled)

5. A lactic acid bacterium introduced with the vector according to claim 3.

6. (canceled)

7. (canceled)

8. A lactic acid bacteria composition comprising: the lactic acid bacterium according to claim 5.

9. The lactic acid bacteria composition according to claim 8, which is fermented milk.

10. The lactic acid bacteria composition according to claim 8, further comprising: an exopolysaccharide derived from the lactic acid bacterium.

11. A method for producing fermented milk, comprising: a fermentation step of adding the lactic acid bacterium according to claim 5 to a milk preparation solution containing raw material milk and fermenting the mixture.

12. A method for improving the viscosity of fermented milk, comprising: a fermentation step of adding the lactic acid bacterium according to claim 5 to a milk preparation solution containing raw material milk and fermenting the mixture.

13. A method for evaluating lactic acid bacteria for presence or absence of a fermented milk viscosity improving effect using as an index at least one selected from the group consisting of DNAs encoding the following proteins (a) to (d):
(a) a protein which is composed of an amino acid sequence set forth in SEQ ID NO: 1;
(b) a protein which is, in the amino acid sequence set forth in SEQ ID NO: 1, composed of an amino acid sequence in which one or more amino acids other than tyrosine at position 40 are substituted, deleted, inserted, and/or added and has a fermented milk viscosity improving effect;
(c) a protein which is composed of an amino acid sequence having 80% or more identity with the amino acid sequence set forth in SEQ ID NO: 1, has tyrosine as an amino acid corresponding to position 40 of the amino acid sequence set forth in SEQ ID NO: 1, and has a fermented milk viscosity improving effect; and
(d) a protein which is composed of an amino acid sequence encoded by a DNA that hybridizes under stringent conditions with a complementary strand of a DNA composed of the nucleotide sequence set forth in SEQ ID NO: 2, has tyrosine as an amino acid corresponding to position 40 of the amino acid sequence set forth in SEQ ID NO: 1, and has a fermented milk viscosity improving effect.

14. Fermented milk comprising: a lactic acid bacterium evaluated as having a fermented milk viscosity improving effect by the method for evaluating lactic acid bacteria according to claim 13.

15. A method for producing lactic acid bacteria, comprising:
an evaluation step of evaluating whether or not the lactic acid bacteria have a fermented milk viscosity improving effect by the method for evaluating lactic acid bacteria according to claim 13; and
a step of obtaining lactic acid bacteria evaluated as having a fermented milk viscosity improving effect in the evaluation step.

16. A method for producing fermented milk, comprising:
an evaluation step of evaluating whether or not the lactic acid bacteria have a fermented milk viscosity improving effect by the method for evaluating lactic acid bacteria according to claim 13; and
a fermentation step of adding the lactic acid bacteria evaluated as having a fermented milk viscosity improving effect in the evaluation step to a milk preparation solution containing raw material milk and fermenting the mixture.

17. A method for improving the viscosity of fermented milk, comprising:
an evaluation step of evaluating whether or not the lactic acid bacteria have a fermented milk viscosity improving effect by the method for evaluating lactic acid bacteria according to claim 13; and
a fermentation step of adding the lactic acid bacteria evaluated as having a fermented milk viscosity improving effect in the evaluation step to a milk preparation solution containing raw material milk and fermenting the mixture.

18. A fermented milk thickener comprising: an exopolysaccharide derived from the lactic acid bacterium according to claim 5 as an active ingredient.

19. A method for producing an exopolysaccharide of lactic acid bacteria, comprising: a step of adding the lactic acid bacterium according to claim 5 to a medium containing glucose and/or a saccharide composed of glucose, fermenting the mixture, and collecting an exopolysaccharide contained in a fermented product.

20. A method for producing an exopolysaccharide of lactic acid bacteria, comprising:
an evaluation step of evaluating whether or not the lactic acid bacteria have a fermented milk viscosity improving effect by the method for evaluating lactic acid bacteria according to claim 13; and
a step of adding the lactic acid bacteria evaluated as having a fermented milk viscosity improving effect in the evaluation step to a medium containing glucose and/or a saccharide composed of glucose, fermenting the mixture, and collecting an exopolysaccharide contained in a fermented product.

21. A method for producing a fermented milk thickener, comprising:
a fermentation step of adding the lactic acid bacterium according to claim 5 to a medium containing glucose and/or a saccharide composed of glucose, fermenting the mixture, and obtaining a fermented product containing an exopolysaccharide; and
a step of obtaining a fermented milk thickener containing the exopolysaccharide as an active ingredient.

22. A method for producing a fermented milk thickener, comprising:
an evaluation step of evaluating whether or not the lactic acid bacteria have a fermented milk viscosity improving effect by the method for evaluating lactic acid bacteria according to claim 13;
a fermentation step of adding the lactic acid bacteria evaluated as having a fermented milk viscosity improving effect in the evaluation step to a medium containing glucose and/or a saccharide composed of glucose, fermenting the mixture, and obtaining a fermented product containing an exopolysaccharide; and
a step of obtaining a fermented milk thickener containing the exopolysaccharide as an active ingredient.
